(19) United States
(12) Patent Application Publication
(10) Pub. No.: US 2015/0299280 A1
(43) Pub. Date: Oct. 22, 2015

(54) MEDICAL TREATMENT USE OF CELL-MEMBRANE-PERMEABLE FIBROBLAST GROWTH FACTOR

(71) Applicant: NATIONAL INSTITUTE OF RADIOLOGICAL SCIENCES, Chiba-shi, Chiba (JP)

(72) Inventors: Fumiaki NAKAYAMA, Chiba (JP); Sachiko UMEDA, Chiba (JP); Takeshi YASUDA, Chiba (JP); Mayumi FUJITA, Chiba (JP); Takashi IMAI, Chiba (JP)

(73) Assignee: NATIONAL INSTITUTE OF RADIOLOGICAL SCIENCES, Chiba-shi, Chiba (JP)

(21) Appl. No.: 14/647,634

(22) PCT Filed: Nov. 11, 2013
(86) PCT No.: PCT/JP2013/080382

§ 371 Date: May 27, 2015

(30) Foreign Application Priority Data
Nov. 28, 2012 (JP) 2012-259816

(51) Int. Cl. C07K 14/50; C07K 14/705

(52) U.S. Cl. CPC C07K 14/50; C07K 14/705; A61K 38/00

(57) ABSTRACT

The present invention relates to a chimeric protein formed by fusing a CPP containing a CPP-C domain in any of an FGF11, FGF12, FGF13, and FGF14 to an FGF1 or an FGF2, or DNA molecules that contain DNA sequences coding the FGF1 or the FGF2 and DNA sequences coding a CPP-C or vectors containing these DNA sequences. These chimeric protein, DNA molecules, or vectors can be used for a medicine or a method of treatment effective to a tissue on which an expression of an FGFR is low or becomes low due to any cause, a medicine or a method of treatment that can further dynamize bioactivity of the FGF1 or the FGF2 via the FGFR, or a medicine or a method of treatment that protects a stem cell against an influence from radiation exposure, a chemotherapy, or a similar treatment.

### TECHNICAL FIELD

The present invention relates to a cell-membrane-permeable fibroblast growth factor. More specifically, the present invention relates to a chimeric protein formed by fusing a cell-membrane-permeable peptide (hereinafter abbreviated as a CPP) to a fibroblast growth factor (hereinafter abbreviated as an FGF) or a medical treatment use or a cell culture use of the chimeric protein.

### BACKGROUND ART

The FGF is a physiologically active substance that stimulates a cell proliferation of mammals. Currently, 23 members classified into seven subfamilies are identified. Most members of the FGF interact with a fibroblast growth factor receptor (hereinafter abbreviated as an FGFR) and activate a tyrosine kinase in an intracellular domain. Through a signal transduction generated by the activation, bioactivity is provided (see Introductions in Non-Patent Documents 1 to 24 or a similar document). FGFR family includes four kinds of FGFR1 to FGFR4. FGFR1 to FGFR3 each has subgroups: FGFR1a, FGFR1b and FGFR1c, FGFR2a, FGFR2b and FGFR2c, and FGFR3a, FGFR3b, and FGFR3c (for example, Non-Patent Documents 1 and 17). It has been known that the b subgroups are expressed on an epithelial tissue or a similar tissue while the c subgroups are expressed on a mesenchymal tissue or a similar tissue (for example, Non-Patent Documents 1 and 17).

The FGF1 (may also be referred to as an acidic fibroblast growth factor) belongs to an identical subfamily to the FGF2 (may also be referred to as a basic fibroblast growth factor) (an FGF1 subfamily), having a similar bioactivity to the FGF2. While the FGF2 weakly interacts with the FGFR2b that are specifically expressed on the epithelial cell, the FGF1 has a feature of allowing an interaction with all FGFRs (Non-Patent Document 1). It has been known that the FGF1 also interacts with CSNK2B, CSNK2A2, HSPA9, S100A13, casein kinase 2, and FIBP (Non-Patent Documents 25 to 29). In view of this, the FGF1 has a possibility of involving various physiological activities at various mesodermally derived tissues and neuroectodermal tissues, such as the brain, the eyes, the kidney, the placenta, and the adrenal tissue, not only at a stage of development but also for the adult. The FGF1 has been examined on a treatment of ischemic heart disease (Non-Patent Document 11), vascularization for critical limb ischemia (Non-Patent Document 12), a healing of skin ulcer of a diabetic mouse (Non-Patent Document 13), a treatment of tympanic membrane perforation (Non-Patent Document 14), a prevention and a treatment of radiation-induced intestinal damage (Non-Patent Document 2), a prevention of radiation-induced hair follicle damage (Non-Patent Document 15), a maintenance of stem cells (Non-Patent Document 16), a reduction in migration and invasion of cancer cells (Non-Patent Document 17), or a similar case.

Meanwhile, different from the FGF2, the FGF1 is unstable if not forming a complex with a heparin and a heparan sulfate (HS), failing to provide the bioactivity. To improve such property, there has been made an attempt to substitute some amino acids in the FGF1 to stabilize the structure (Non-Patent Documents 9, 10, and 19). However, currently, the FGF1 actually launched as a medicinal product is a wound healing agent (generic name: trafermin) whose active ingredient is the FGF2 and a medicine for the prevention and treatment of radiochemotherapy-induced oral mucositis whose active ingredient is an FGF7 (generic name: palifermin).

Some detailed reports on the mechanism of action of the FGF1 have been released. It is reported that, to provide the biological activity, such as a cell division and a cell proliferation, by the FGF1, as well as the signal transduction by the interaction with the FGFR, translocation of the FGF1 into the nucleus is necessary (Non-Patent Documents 3 to 5). For example, Wiedlocha and et al., have reported that, in the experiment using the FGF1 labelled with CAAX, the translocation of the FGF1 into the nucleus to stimulate DNA synthesis and the translocation of the FGFR1 into the cells require a binding of the FGF1 to the FGFR (Non-Patent Document 3). Imamura and et al., report the following. The Imamura and et al., added the FGF1 lacking the nuclear localization sequence and the FGF1 recovering the nuclear localization sequence to the LE-II cell under the conditions of allowing the interaction with the FGFR. Consequently, the FGF1 lacking the nuclear localization sequence failed to provide the cell division activity. Meanwhile, the FGF1 having the nuclear localization sequence has the cell division activity (Non-Patent Document 5). Wiedlocha and et al., also report the following. Wiedlocha and et al., fused the diphtheria toxin A to the FGF1 to form the chimeric protein and translocate this chimeric protein into the cells via the diphtheria toxin A receptor. Consequently, the DNA synthesis was promoted (Non-Patent Document 4). This report suggests that the translocation of the FGF1 into the nucleus has some sort of relationship with the cell division activity or the cell proliferation. However, this also suggests that the translocation of the FGF1 into the cells without via the FGFR only to result in the synthesis of DNA. In this report, Wiedlocha and et al., conclude that the activation of the tyrosine kinase with the FGFR would be required for another process related to the cell division and the cell proliferation.

Wiedlocha and et al., also report the following. The chimeric protein formed by fusing the diphtheria toxin A, which is one kind of the CPP, to the FGF1 is translocated into the cells via the diphtheria toxin A receptor under the condition of lacking the heparin. However, under the presence of the heparin, the chimeric protein is not translocated into the cells. Thus, Wiedlocha and et al., teach that the heparin prevents the chimeric protein formed by fusing the CPP from passing through the cell membrane (Non-Patent Documents 4 and 24).

Regarding the mechanism of action of the FGF2, similar to the FGF1, Non-Patent Document 23 or a similar document reports the signal transduction and the cellular internalization via the FGFR and the action inside the cells.

The examinations on the pharmacological or biological activity of the FGF1 or FGF2 up to the present suppose such mechanism of action of the FGF1 or FGF2. That is, on the supposition that the FGFR is expressed on cells at a lesion site or cells of a damaged tissue, the FGF1 or the FGF2 is interacted with the FGFR. Then, through the signal transduction and the cellular internalization of the FGF1 or FGF2 via the FGFR, a desired activity is attempted to be generated. However, currently, the mechanism of action of the anti-apoptotic effect is not clear.

For example, Meyer and et al., report that, with the keratinocyte lacking the FGFR1 and 2, the migration of the keratinocyte slows, delaying wound skin. Meyer and et al., conclude that the presence of the FGFR1 or FGFR2 is necessary to heal the wound skin (Non-Patent Document 22).

Hagiwara and et al., report the following. In relation to the profile of the FGFR expression on the jejunum before and after irradiation, the FGF1 is superior to the other FGF family members in the prevention and the treatment of the damage of the intestinal tract induced by radiation (Non-Patent Document 2).

Palmen and et al., report the following. The FGF1 is effective to recover functions from an ischemic heart disease. The intracellular signal transduction system via the FGFR brings this effect (Non-Patent Document 11). Nikol and et al., report the following. NV1FGF was administered to the muscle of the patient with critical ischemic limb to locally express the NV1FGF. This significantly reduced the risk of amputation (Non-Patent Document 12). However, Palmen and et al., report that the administration group had no significant in the healing of ulcer from the non-administration group.

Goldman and et al., report the test result of administration of the FGF1 to the perforated eardrum (Non-Patent Document 14).

Mellin and et al., report the following. The administration of the FGF1 to the skin ulcer of the diabetic mouse promoted the healing of wound dependent of the dose (Non-Patent Document 13).

Chen and et al., report the following. The ERK1/2 phosphorylation expression indicative of the activation of the FGFR by the FGF and the NANOG expression indicative of pluripotency are taken as indexes. The thermally-stable mutation FGF1 into which the substitution of three amino acids, Q40P, S471, and H93G; is introduced maintains the self-renewal ability and the pluripotency of the ES cells and the iPS cells (Non-Patent Document 16).

Liu and et al., report the following. Liu and et al., focus on the point that on tumor cells, the FGFR1c is dominantly expressed while the expression of the FGFR1b is low. On the pancreatic cancer cell line, the FGFR1b is forcibly overexpressed, and then the FGF1 or a similar factor is administered. This inhibits the proliferation, the migration, and the invasion of the cancer cells (Non-Patent Document 17).

Nakayama and et al., report the following. Nakayama and et al., administered the FGF1 to the depilated skin of the BALB/c mouse whose hair follicle was induced to the growth phase by depilation. Then, irradiation induced the apoptosis of the hair follicle cells. This resulted in a reduction in apoptosis (Non-Patent Document 15). Fu and et al., report the following. Fu and et al., injected the FGF1, the FGF1 lacking the nuclear localization domain (28-154), or a similar factor to the animal model. The FGF1 lacking the nuclear localization domain increased the anti-apoptotic effect more than the FGF1 having the identical domain (Non-Patent Document 21). Meanwhile, Rodriguez and et al., report the following. Rodriguez and et al., introduced the FGF1 expression vectors into the PC12 cell. When Rodriguez and et al., observed the nuclear localization of the FGF1 by the test where the FGF1 was expressed in the cells with dexamethasone, the neuronal differentiation and the anti-apoptotic capacity were increased (Non-Patent Document 20).

Thus, it is reported that the anti-apoptotic effect occurs by the translocation of the FGF1 into the nucleus regardless of the interaction with the FGFR. Meanwhile, it is also reported that after the FGF1 is translocated into the cells by the interaction with the FGFR, the anti-apoptotic effect increases when the FGF1 is not translocated into the nucleus rather than the FGF1 migrating into the nucleus. Therefore, currently, the mechanism of action of the anti-apoptotic effect brought by the FGF1 is not clear. However, in the test for confirming the anti-apoptotic effect of the FGF1 is usually conducted under the condition supposing the interaction between the FGF1 and the FGFR.

The FGF11 subfamily member, different from the other FGF family members including the FGF1 and 2, has a unique property of not interacting with the FGFR. The FGF11 to 14 belong to this subfamily, and the amino acid sequences for the FGF11 to 14 have also been known (Patent Documents 1 to 6). However, how these FGFs can be translocated into the cells or whether the FGF is involved in some sort of physiological action in the cells or not was not clear well (Non-Patent Document 24).

The inventors report the following. The FGF12 can be translocated from the outside of the cells into the cells independent of the FGFR. The cell-membrane-permeable peptide domains (hereinafter may be abbreviated as CPP domains) in charge of the cellular internalization are present at two positions, the center (hereinafter may be referred to as a CPP-M domain) and a C-terminal part (hereinafter may be referred to as a CPP-C domain) (Non-Patent Document 8). This report described that a similar domain is present in another member of the FGF11 subfamily; however, the CPP-C domain is not present at the FGF1. This domain promotes the cellular internalization of the FGF12. This report also described the following. The peptide consisted of the CPP-C domain of the FGF12 is fused to the FGF1. The obtained chimeric protein can be translocated into the cells independent of the FGFR.

The inventors also described that the FGF12 itself has the anti-apoptotic activity. Further, the FGF12 fragment lacking the amino acid residues 140-181 lacks the cellular internalization property and the anti-apoptotic activity. However, adding TAT to the FGF12 fragment to recover the cellular internalization property also remarkably reduced the radiation-induced apoptosis (Non-Patent Document 8). In the subsequent study, the inventors reported the following. By the intracellular expression, the peptide consisted of 30 amino acids derived from FGF12 containing any of the CPP-M domain or the CPP-C domain proliferated and differentiated the small intestinal epithelial cells and reduced the apoptosis (Non-Patent Document 18).

### CITATION LIST

### Patent Document

Patent Document 1: Japanese Unexamined Patent Application Publication No. 2000-509965
Patent Document 2: Japanese Unexamined Patent Application Publication No. 2001-507561
Patent Document 3: Japanese Unexamined Patent Application Publication No. 11-508125
Patent Document 4: Japanese Unexamined Patent Application Publication No. 2001-505526
Patent Document 5: Japanese Unexamined Patent Application Publication No. 11-506917
Patent Document 6: Japanese Unexamined Patent Application Publication No. 2000-517187
Patent Document 7: Japanese Unexamined Patent Application Publication No. 2003-518944
Patent Document 8: Japanese Unexamined Patent Application Publication No. 11-507504
Patent Document 9: Japanese Unexamined Patent Application Publication No. 2003-052387

### Non-Patent Document

[Non-Patent Document 1] Ornitz D M, Xu J, Colvin J S, McEwen D G MacArthur C A, Coulier F, Gao G, Goldfarb M. Receptor specificity of the fibroblast growth factor family. J. Biol. Chem. 1996; 271:15292-15297.
[Non-Patent Document 2] Hagiwara A, Nakayama F, Motomura K, Asada M, Suzuki M, Imamura T, Akashi M. Comparison of expression profiles of several fibroblast growth factor receptors in the mouse jejunum: suggestive evidence for a differential radioprotective effect among major FGF family members and the potency of FGF1. Radiat. Res. 2009; 172:58-65.
[Non-Patent Document 3] Wiedlocha A, Falnes P O, Rapak A, Klingenberg O, Munoz R, Olsnes S. Translocation of cytosol of exogenous, CAAX-tagged acidic fibroblast growth factor. J. Biol. Chem. 1995; 270:30680-30685.
[Non-Patent Document 4] Wiedlocha A, Falnes P O, Madshus I H, Sandvig K, Olsnes S. Dual mode of signal transduction by externally added acidic fibroblast growth factor. Cell 1994; 76:1039-1051,
[Non-Patent Document 5] Imamura T, Engleka K, Zhan X, Tokita Y, Forough R, Roeder D, Jackson A, Maier J A, Hla T, Maciag T. Recovery of mitogenic activity of a growth factor mutant with a nuclear translocation sequence. Science 1990; 249:1567-1570.
[Non-Patent Document 6] Klingenberg O, Widlocha A, Rapak A, Munoz R, Falnes P, Olsnes S. Inability of the acidic fibroblast growth factor mutant K132E to stimulate DNA synthesis after translocation into cells. J. Biol. Chem. 1998; 273:11164-11172.
[Non-Patent Document 7] Olsen S K, Garbi M, Zampieri N, Eliseenkova A V, Ornitz D M, Goldfarb M, Mohammadi M. Fibroblast growth factor (FGF) homologous factors share structural but not functional homology with FGFs. J. Biol. Chem. 2003; 278:34226-34236.
[Non-Patent Document 8] Nakayama F, Yasuda T, Umeda S, Asada M, Imamura T, Meineke V, Akashi M. Fibroblast growth factor-12 (FGF12) translocation into intestinal epithelial cells is dependent on a novel cell-penetrating peptide domain: involvement of internalization in the in vivo role of exogenous FGF12. J. Biol. Chem. 2011; 286:25823-25834.
[Non-Patent Document 9] Zakrzewska M, Krowarsch D, Wiedlocha A, Olsnes S, Otlewski J. Highly stable mutants of human fibroblast growth factor-1 exhibit prolonged biological action. J. Mol. Biol. 2005; 352:860-875.
[Non-Patent Document 10] Zakrzewska M, Wiedlocha A, Szlachcic A, Krowarsch D, Otlewski J, Olsnes S. Increased protein stability of FGF1 can compensate for its reduced affinity for heparin. J. Biol. Chem. 2009; 284:25388-25403.
[Non-Patent Document 11] Palmen M, Daemen M J, De Windt L J, Willems J, Dassen W R, Heeneman S, Zimmermann R, Van Bilsen M, Doevendans P A. Fibroblast growth factor-1 improves cardiac functional recovery and enhances cell survival after ischemia and reperfusion: a fibroblast growth factor receptor, protein kinase C, and tyrosine kinase-dependent mechanism. J. Am. Coll. Cardiol. 2004; 44:1113-1123.
[Non-Patent Document 12] Nikol S, Baumgartner I, Van Belle E, Diehm C, Visona A, Capogrossi M C, Ferreira-Maldent N, Gallino A, Wyatt M G; Wijesinghe L D, Fusari M, Stephan D, Emmerich J, Pompilio Vermassen F, Pham E, Grek V, Coleman M, Meyer F. Therapeutic angiogenesis with intramuscular NV1FGF improves amputation-free survival in patients with critical limb ischemia. Mol. Ther. 2008; 16:972-978.
[Non-Patent Document 13] Mellin T N, Cashen D E, Ronan J J, Murphy B S, DiSalvo J, Thomas K A. Acidic fibroblast growth factor accelerates dermal wound healing in diabetic mice. J. Invest. Dermatol. 1995; 104:850-855.
[Non-Patent Document 14] Goldman S A, Siegfried J, Scoleri P, Aydogen L B, Cass S P. The effect of acidic fibroblast growth factor and live yeast cell derivative on tympanic membrane regeneration in a rat model. Otolaryngol. Head Neck Surg. 1997; 117:616-621.
[Non-Patent Document 15] Nakayama F, Hagiwara A, Kimura M, Akashi M, Imamura T. Evaluation of radiation-induced hair follicle apoptosis in mice and the preventive effects of fibroblast growth factor-1. Exp. Dermatol. 2009; 18:889-892.
[Non-Patent Document 16] Chen G Gulbranson D R, Yu P, Hou Z, Thomson J A. Thermal stability of fibroblast growth factor protein is a determinant factor in regulating self-renewal, differentiation, and reprogramming in human pluripotent stem cells. Stem Cells 2012; 30:623-630.
[Non-Patent Document 17] Liu Z, Neiss N, Zhou S, Henne-Bruns D, Korc M, Bachem M, Kornmann M., Identification of a fibroblast growth factor receptor 1 splice variant that inhibits pancreatic cancer cell proliferation, Cancer Res. 2007; 67:2712-2719.
[Non-Patent Document 18] Nakayama Fumiaki, Sadako Umeda, Yasuda Takeshi, Asada Masahiro, Suzuki Osamu, Imamura Tow, Imai Takashi, The 54th Annual Meeting of the Japanese Radiation Research Society Summary, Fibroblast Growth Factor-12 Translocation into Intestinal Epithelial Cells is Dependent on a Novel Cell-Penetrating Peptide Domain: Involvement of Internalization in the in Vivo Role of Exogenous FGF12, 2011
[Non-Patent Document 19] Fumiaki Nakayama et al., Structural Stability of Human Fibroblast Growth Factor-1 Is Essential for Protective Effects Against Radiation-Induced Intestinal Damage, International Journal of Radiation Oncology Biology Physics, pp. 1-7, 2012.
[Non-Patent Document 20] Aida Rodriguez-Enfedaque et al., FGF1 nuclear translocation is required for both its neurotrophic activity and its p53-dependent apotosis protection, Biochimica et Biophysica Acta, 1793 (2009) 1710-1727.
[Non-Patent Document 21] Xiao-Bing Fu et al., Enhanced anti-apoptosis and gut epithelium protection function of acidic fibroblast growth factor after cancelling of its mitogenic activity, World J Gastroenterol 2004; 10(24):3590-3596.
[Non-Patent Document 22] Michael Meyer et al., FGF receptors 1 and 2 are key regulators of keratinocyte migration in vitro and in wounded skin, Journal of Cell Science, 2012.
[Non-Patent Document 23] Biochemistry, 81th volume, seventh issue, page 592 to page 596, July, 2009
[Non-Patent Document 24] Slur Olsnes, et al., Transport of Exogenous Growth Factors and Cytokines to the Cytosol and to the Nucleus, Physiol Rev, Vol. 83, January 2003.
[Non-Patent Document 25] Skjerpen, Camilla Skiple; Nilsen Trine, Wesche Jorgen, Olsnes Sjur, “Binding of FGF-1 variants to protein kinase CK2 correlates with mitogenicity”. EMBO J. (England) 21 (15): 4058-69, August 2002.
[Non-Patent Document 26] Mizukoshi, E; Suzuki M, Loupatov A, Uruno T, Hayashi H, Misono T, Kaul S C, Wadhwa R, Imamura T (October 1999). “Fibroblast growth factor-1 interacts with the glucose-regulated protein GRP75/mortalin”. Biochem. J. (ENGLAND) 343 (2): 461-6.
[Non-Patent Document 27] Mouta Carreira, C; LaVallee T M, Tarantini F, Jackson A, Lathrop J T, Hampton B, Burgess W H, Maciag T (August 1998). “S100A13 is involved in the regulation of fibroblast growth factor-1 and p40 synaptotagmin-1 release in vitro”. J. Biol. Chem. (UNITED STATES) 273 (35): 22224-31.
[Non-Patent Document 28] Landriscina, M; Bagala C, Mandinova A, Soldi R, Micucci I, Bellum S, Prudovsky I, Maciag T (July 2001). “Copper induces the assembly of a multiprotein aggregate implicated in the release of fibroblast growth factor 1 in response to stress”. J. Biol. Chem. (United States) 276 (27): 25549-57.
[Non-Patent Document 29] Kolpakova, E; Wiedlocha A, Stenmark H, Klingenberg O, Falnes P O, Olsnes S (November 1998). “Cloning of an intracellular protein that binds selectively to mitogenic acidic fibroblast growth factor”. Biochem. J. (ENGLAND) 336 (Pt 1): 213-22.

### SUMMARY OF INVENTION

### Technical Problem

As described above, it is considered that the FGF1 and FGF2 basically provide the bioactivity through the signal transduction and the cellular internalization via the interaction with the FGFR. Factors such as the expression level and the expression profile of the FGFR on the cell surface targeted for the interaction also affect the bioactivity of the FGF1 and the FGF2. Therefore, the blood system cells, such as the lymphocyte, on which the expression of the FGFR is originally low and a tissue on which the expression of the FGF receptor lowers due to various factors, such as a burn, radiation, a deficiency of blood supply, and an infection, the FGF1 and the FGF2 cannot fully provide the physiological action.

In this respect, Hagiwara and et al., report that, after the total body irradiation with gamma rays, the expression level of the FGFR2b temporarily deteriorates at the jejunum of the mouse (Non-Patent Document 2). Mellin and et al., point out that a transcription level of the FGFR deteriorates at the diabetic skin ulcer model, causing a delay in the healing of wound (Non-Patent Document 13). However, both reports do not present solutions for the problems associated to the low expression of the FGFR. Therefore, except for forcibly expressing the FGFR on the tumor cells (Non-Patent Document 17), the report presenting the means for fundamentally solving this problem has not yet found. The FGF1 cannot provide the effect on the hematopoietic cells, such as the lymphocyte and the granulocyte, which express a low level of FGFR. However, the above-described reports do not mention this problem itself at all.

If a means can be provided to inhibit a proliferation and a metastasis of tumor cells without forcibly expressing the FGFR, it is apparent that such means is superior to the conventional techniques. In the case where the FGFR is expressed on the cell surface, the FGF1 or the FGF2 can be translocated into the cells via the FGFR, and the signals can be transduced via the FGFR. It is considered that this eliminates the need for translocating the FGF1 or a similar factor into the cells through another route. However, if a means can be provided to enhance the bioactivity of the FGF1 or the FGF2 via the FGFR, this will be beneficial. Up to the present, there seems to have been no reports on the use of the FGF1 or the FGF2 as a means for protecting stem cells from an influence, such as irradiation or chemotherapy. Therefore, when such treatment can be achieved with the FGF1 or the FGF2, novel choices can be provided for promoting a recovery after a treatment by radiotherapy or chemotherapy or reducing the side-effects.

### Solutions to Problem

The inventors of the present invention created the chimeric protein by fusing the CPP-C of FGF12 to the FGF1 and transferred it into the non-FGFR-expressing cells by contact with the cells. Unexpectedly, the inventors have found that various biological or pharmacological activities of the FGF1 can be expressed in them. The inventors found that such chimeric protein can inhibit the proliferation and the metastasis of the tumor cells without forcibly expressing the FGFR. Unexpectedly, the inventors have found that the chimeric protein provides higher biological or pharmacological activities in the FGFR-expressing cells than the native FGF1 by contact with the cells. Further, the inventors have found that such chimeric protein can protect the stem cells from the radiotherapy and the chemotherapy. This knowledge is thought to be similarly applicable to the FGF2, which belongs to the identical subfamily to the FGF1. The present invention is based on this knowledge.

That is, the present invention provides the chimeric protein formed by fusing the CPP containing any CPP-C domain of FGF11, FGF12, FGF13, or FGF14 to FGF1 or FGF2 in one embodiment of the present invention.

The present invention provides DNA molecules that contain DNA sequences coding the FGF1 or the FGF2 and DNA sequences coding the CPP-C or vectors containing these DNA sequences in another embodiment.

The present invention provides a medicinal composition whose active ingredient is the chimeric protein, the DNA molecules, or the vectors in yet another embodiment.

The present invention also provides methods for preventing or treating various diseases or symptoms caused by a physiological phenomenon involving FGF1 or FGF2 in yet another embodiment. The methods include an administration step of administrating the chimeric protein, the DNA molecule, the vector, or the composition of a therapeutically effective amount to a target requiring the chimeric protein, the DNA molecule, the vector, or the composition.

The present invention also provides a use of the chimeric protein, the DNA molecule, the vector, or the composition for preparing a medicine or a cell culture medium in yet another embodiment.

The methods, the medicinal compositions, the chimeric proteins, or similar conditions according to the present invention are not limited to these. However, for example, the methods, the medicinal compositions, the chimeric proteins, or similar conditions can be used to: maintain or grow a cell, protect a stem cell, inhibit an apoptosis of a cell, promote a migration of a cell, inhibit a proliferation or a metastasis of a tumor cell, or recover a function of an ischemic tissue. More specifically, the methods, the medicinal compositions, or the chimeric proteins of the present invention are effective to, for example, promote the healing of wound, prevent or treat damage of the intestinal tract induced by radiation and chemotherapy, prevent or treat alopecia induced by radiation and chemotherapy, treat a limb ischemia, treat a diabetic skin ulcer and a diabetic gangrene, prevent and treat an ischemic coronary artery disease, treat a tympanic membrane perforation, and inhibit a proliferation and a metastasis of malignant tumors.

The CPP-FGF1 or the CPP-FGF2 chimeric protein used as the active ingredient in the present invention can be translocated into the cells more efficiently than the native FGF1 or FGF2. However, it is thought that the highly-efficient translocation into the cells does not go through the FGFR. Conventionally, the various biological or pharmacological activities brought by the FGF1 or the FGF2 are not expressed only by simply translocating the FGF1 or a similar factor into nucleus. Therefore, it was thought that the cellular internalization of the FGF1 or a similar factor through the FGFR and the signal transduction through the FGFR were necessary. However, the CPP-FGF1 or CPP-FGF2 chimeric protein, which is used as the active ingredient in the present invention, can provide the various biological or pharmacological activities brought by the FGF1 or the FGF2, although the CPP-FGF1 or CPP-FGF2 chimeric protein is translocated into the cells without via the FGFR. Therefore, the medicinal composition according to the present invention is especially beneficial for the treatment or the prevention of a symptom or a disease where all of or some of the FGFRs are not expressed or the FGFR is expressed at a low level on a lesion or cells of a damaged tissue to be treated, a symptom where the FGF1 or the FGF2 cannot be translocated into the cells for any reason, or a symptom where the FGF1 or a similar factor cannot interact with the FGFR. In such symptoms, the native FGF1 or a similar factor cannot fully provide the biological or pharmacological activity. However, the present invention can provide the means for fundamentally solving the problems.

The present invention provides novel means for inhibiting the proliferation and the metastasis of the tumor cells. The tumor cells exhibit a low expression level by the FGFR1b; therefore, a treatment using the native FGF1 or FGF2 fails to obtain the sufficient curative effect. In view of this, the treatment using the FGF up to the present is that FGF1 is administered against the tumor cells forcibly expressing the FGFR1b. However, the present invention eliminates the need for forcibly expressing the FGFR1b on the tumor cell. Only administrating the medicinal composition of the present invention can obtain the curative effect. It is thought that the mechanism of action that the CPP-FGF1 or the CPP-FGF2 chimeric protein can be translocated into the cells independent of the FGFR1b brings such efficacy. This is thought to allow inhibiting the proliferation and the metastasis of the tumor cells over the wide range.

The present invention also provides means for providing efficacy for a symptom or a disease where the lesion or the cells of the damaged tissue express(es) the FGFR more than the conventional method using the FGF1 or the FGF2. In the symptom or the disease where the FGFR is expressed on the cell surface, the FGF1 or the FGF2 is caused to be translocated into the cells via the FGFR and the signal transduction can be generated via the FGFR. Accordingly, it is thought that there is no need to translocate the FGF1 or the FGF2 into the cells through another route. Meanwhile, the CPP-FGF1 or CPP-FGF2 chimeric protein unexpectedly provides the biological or pharmacological activity higher than the native FGF1 or FGF2.

The CPP-FGF1 or the CPP-FGF2 chimeric protein also has an effect to be able to protect the stem cells against irradiation and the chemotherapy. In view of this, the present invention provides new choices to promote recovery after the treatments by radiotherapy and chemotherapy and to reduce the side-effects.

Here, the following summarizes the meanings of the abbreviations and terms used in this description.

FGF: fibroblast growth factor (Note that this description may collectively call the fibroblast growth factor including a mutant, which will be described later, and the chimeric protein).
FGF1: fibroblast growth factor 1 (Note that this description may collectively call the fibroblast growth factor 1 including the mutant, which will be described later.)
FGF2: fibroblast growth factor 2 (Note that this description may collectively call the fibroblast growth factor 2 including the mutant, which will be described later.)
FGF11: fibroblast growth factor 11 (Note that this description may collectively call the fibroblast growth factor 11 including the mutant, which will be described later.)
FGF12: fibroblast growth factor 12 (Note that this description may collectively call the fibroblast growth factor 12 including the mutant, which will be described later.)
FGF13: fibroblast growth factor 13 (Note that this description may collectively call the fibroblast growth factor 13 including the mutant, which will be described later.)
FGF14: fibroblast growth factor 14 (Note that this description may collectively call the fibroblast growth factor 14 including the mutant, which will be described later.) Mutant: the FGF1 represented by any of amino acid sequences shown by SEQ ID NOs. 1 to 5, the FGF2 represented by any of amino acid sequences shown by SEQ ID NOs. 6 to 10, or a protein or a peptide where some amino acids of a cell-membrane-permeable peptide represented by any of amino acid sequences shown by SEQ ID NOs. 11 to 29 are substituted or deleted, or one or more amino acids are added; or the other known FGF1 or FGF2 or the other known protein or peptide where some amino acids of a cell-membrane-permeable peptide are substituted or deleted, or one or more amino acids are added.
CPP: cell-membrane-permeable peptide
CPP-C domain: cell-membrane-permeable peptide domain present at a C-terminal region of an FGF11 subfamily member
CPP-M domain: cell-membrane-permeable peptide domain present at a center of the
FGF11 subfamily member
CPP-C: unless otherwise mentioned, the FGF11 subfamily CPP-C domain or a peptide that contains the amino acid sequence where some of amino acids are substituted or deleted and has membrane permeability
CPP-FGF1 chimeric protein: chimeric protein formed by fusing the CPP-C to the FGF1
CPP-FGF2 chimeric protein: chimeric protein formed by fusing the CPP-C to the FGF2
The CPP-FGF1 chimeric protein and the CPP-FGF2 chimeric protein may be simply collectively called as the chimeric protein.
FGFR: fibroblast growth factor receptor
FACS: flow cytometry
Hydrophilic amino acid: This description uses the hydrophilic amino acid including at least an arginine, an aspartic acid, a glutamic acid, a histidine, and a lysine.
Hydrophobic amino acid: This description uses the hydrophobic amino acid including at least an alanine, a cysteine, an isoleucine, a leucine, a methionine, a phenylalanine, a tryptophan, a valine, a proline, and a glycine.
Neutral amino acid: This description uses the neutral amino acid including at least an asparagine, a glutamine, a tyrosine, a threonine, and a serine.

### BRIEF DESCRIPTION OF DRAWINGS

FIG. 1A is a schematic diagram schematically illustrates a structure of a CPP-FGF1 chimeric protein prepared and used in an embodiment of this application.

FIG. 1B illustrates alignments between CPP-C domains of FGF11, FGF12, FGF13, and FGF14. The amino acids shown by italics in FIG. 1B are amino acids different from the corresponding amino acids of the FGF12. FIG. 1C illustrates the alignments between the CPP-C domains of the FGF11, FGF12, FGF13, and FGF14 emphasizing arrangement patterns of the amino acids. In FIG. 1C, the amino acids surrounded by frames are hydrophilic or neutral amino acids, the amino acids shown by italics are hydrophilic amino acids, the amino acids shown by underlines are the neutral amino acids, and the other amino acids are hydrophobic amino acids.

FIG. 2 illustrates histograms obtained by measuring a fluorescence intensity of IEC6 cell lines before addition of and after addition of each fluorescence-labeled FGF by FACS. FIG. 2A illustrates histograms regarding the FGF12B and each fragment of partially cleaved FGF12. FIG. 2B illustrates histograms regarding the FGF1 and each CPP-FGF1 chimeric protein.

FIG. 3 is a graph showing an apoptosis proportion of cells when the IEC6 cell lines are cultured together with each FGF and then are irradiated with X-rays.

FIG. 4A illustrates each FGF12B fragment formed of 30 amino acids derived from different regions of the FGF12B. FIG. 4B is a graph showing the apoptosis proportion of cells when the IEC6 cell lines are cultured together with each FGF12B fragment and then are irradiated with X-rays. FIG. 4C is a graph where a positive fluorescence rate of the IEC6 cell lines after addition of each fluorescence-labeled FGF12B fragment is measured over time by the FACS. FIG. 4D is a graph showing average values of a crypt survival rate of each group when each FGF12B fragment or a saline is administered to an abdominal cavity.

FIG. 5A includes photomicrographies (200 powers) of immunohistostaining by a TUNEL assay on hair follicle bulb regions of mice irradiated with gamma rays on the whole body after the intraperitoneal administration of each FGF or the saline after depilation FIG. 5B is a graph showing average number of apoptosis per hair follicle bulb in each administration group calculated by the TUNEL assay.

FIG. 6A includes photomicrographies (200 powers) of immunohistostaining by the TUNEL assay on crypts in the small intestines of mice whose total bodies were irradiated with gamma rays after intraperitoneal administration of each FGF or the saline. FIG. 6B is a graph showing average number of apoptosis per crypt in each administration group calculated by the TUNEL assay.

FIG. 7A includes photomicrographies (400 powers) of immunohistostaining with an anti-BrdU on cross-sections of small intestines of mice to which BrdU was administered intraperitoneally at 3.5 days after total body irradiation with gamma rays and intraperitoneal administration of each FGF or the saline post-irradiation. FIG. 7B is a graph showing the average values of the crypt survival rate in each administration group.

FIG. 8A includes photomicrographies (200 powers) of the immunohistostaining with an anti-BrdU on cross-sections of small intestinal epithelial tissues of mice to which BrdU was administered intraperitoneally at 3.5 days after total body irradiation with gamma rays and intraperitoneal administration of each FGF or the saline post-irradiation. FIG. 8B is a graph showing average values of crypt length in each administration group.

FIG. 9 includes photomicrographies (400 powers) of the immunohistostaining with an anti-Keratin 15 antibody on tissues in hair follicle bulb regions of mice irradiated with gamma rays after intraperitoneal administration of each FGF after depilation.

FIG. 10A illustrates histograms obtained by measuring a fluorescence intensity of human pancreatic cancer cell lines MIAPaCa-2 and PANC-1 by FACS before and after addition of each fluorescence-labeled FGF. FIG. 10B illustrates graphs showing the relationship between an absorbance (a difference in the absorbance from a control) of formazan that increases in association with a cell proliferation of the human pancreatic cancer cell lines MIAPaCa-2 and PANC-1 and concentrations of the FGF1 and the CPPF2.

FIG. 11A includes photographs of culture mediums when culturing the PANC-1 in culture medium without the addition of FGF or in culture medium supplemented with each FGF and then fixing and staining the PANC-1 with methylene blue/methanol. FIG. 11B is a graph showing average number of colonies in each group stained by the fixed staining shown in FIG. 11A.

FIG. 12 is a graph showing an increase in subcutaneous tumor volume of mice over time when subcutaneously implanting the MIAPaCa-2 to the mice into the thigh and then intraperitoneally administering each FGF or the saline.

FIG. 13A includes photomicrographies (50 powers) of filters where cells invaded to gels by an invasion assay were fixed and stained with a Diff-Quick. FIG. 13B includes graphs showing average values of an invaded cell proportion in each group obtained by the invasion assay.

### DESCRIPTION OF EMBODIMENTS

The present invention relates to a chimeric protein formed by fusing CPP including a CPP-C domain of a FGF11 subfamily member to an FGF1 or an FGF2, DNA molecules containing DNA sequences coding the FGF1 or the FGF2 and DNA sequences coding a CPP-C, or a medicinal composition whose active ingredient is a vector containing theses DNA sequences, and a use of this chimeric protein or a similar compound for medical treatment. The following describes embodiments of the present invention in detail.

### 1. Chimeric Protein

### 1-1. FGF1

The FGF1 is a physiologically active substance known for mammals, such as humans, mice, rats, cattle, and horses. The human FGF1 may have an amino acid sequence represented by SEQ ID NO. 1. The mouse FGF1 may have an amino acid sequence represented by SEQ ID NO. 2. The rat FGF1 may have an amino acid sequence represented by SEQ ID NO. 3. The cattle FGF1 may have an amino acid sequence represented by SEQ ID NO. 4. The horse FGF1 may have an amino acid sequence represented by SEQ ID NO. 5. The present invention may constitute the chimeric protein with the FGF1 derived from any mammal. For example, the FGF1 can be selected according to an animal to be treated.

An identity in the amino acid sequences among the FGF1s for these animals is 90% or more. The sequence identity between the amino acid sequence of the human FGF1 and the amino acid sequences of the FGF1 derived from the other animals is 92% or more. Therefore, even if a mutant whose amino acids differ from some amino acids in the amino acid sequence is used, as long as the mutant is consisted of the amino acid sequences with the sequence identity of 90% or more, the mutant is considered to have a similar biological or pharmacological activity. From this aspect, with respect to the amino acid sequences of any FGF1 represented by SEQ ID NOs. 1 to 5, the mutant is preferably consisted of the amino acid sequences with the sequence identity of 70% or more, more preferably 80% or more, further preferably 90% or more, and especially preferably 95% or more. These mutants are thought: to have the bioactivity similar to the FGF1 before the mutation, or to allow obtaining the functional mutant easily from the mutant. From the similar point, with respect to the amino acid sequence represented by SEQ ID NO. 1, the mutant of the FGF1 used for medical treatment use targeting the human is consisted of the amino acid sequence with the sequence identity of preferably 70% or more, more preferably 80% or more, further preferably 90% or more, and especially preferably 95% or more.

Meanwhile, the amino acid sequence present at an N-terminal region of complete FGF1 contributes to nuclear localization of the FGF1. Regarding at least a part of the biological or pharmacological activity of the cell proliferation or a similar action of the FGF1, the nuclear localization of the FGF1 is thought to be necessary (Non-Patent Documents 3 to 5). Therefore, maintaining the 22th to 28th amino acids in the amino acid sequences represented by SEQ ID NOs. 1 to 5 is preferable. The nuclear localization sequence of this FGF1 can be substituted by a nuclear localization sequence derived from another origin, though. For example, the nuclear localization sequence can be substituted by a nuclear localization sequence derived from a yeast histone 2B (MGKKRKSKAK) or a similar sequence (Non-Patent Document 5). It is thought that even if one to several amino acids of this nuclear localization sequence is substituted by the similar hydrophilic or hydrophobic amino acids, the nuclear localization activity is maintained.

It is thought that substituting the 127 Lys and 133 Lys in the amino acid sequences of SEQ ID NOs. 1 to 5 affects binding of the FGF1 to heparin, activation of the FGFR, or DNA synthesis (Non-Patent Documents 6 and 10). Accordingly, maintaining the amino acids at these positions is also preferable. However, since the chimeric protein of the present invention is comparatively stable, even if the 127 is substituted, a desired activity can be provided.

Amino acid substitution known for stabilization of the stereostructure of the FGF1 or contribution to optimization may be introduced. For example, Gln at the position 55 in the amino acid sequences represented by SEQ ID NOs. 1 to 5 can be substituted by Pro, Ser at the position 62 can be substituted by IIe, His at the position 108 can be substituted by Gly, and Lys at the position 127 can be substituted by Asn, respectively (Non-Patent Documents 9, 10, and 19). Such substitution may be performed on only one amino acid or a plurality of amino acids. However, substituting these amino acids at all positions improves the stability. However, the chimeric protein used for the present invention, as proved by working examples described later, is comparatively stable even without the introduction of such amino acid substitutions, allowing translocation into the cells.

In addition to such substitutions of the amino acids, amino acids other than the amino acids desired to be maintained as described above may be substituted by other amino acids insofar as the above-described sequence identity is kept. Note that the number of amino acids to be substituted is preferably less than 10, more preferably less than 8, and further preferably less than 5.

It is thought that even if the mutant lacks all of or a part of the C-terminal region of the complete FGFR1 or the mutant is the one where another amino acid sequence is inserted in the middle of the identical region and therefore the identical region is separated, the activity of the FGF1 is maintained. Therefore, for example, the mutant may lack all of or some of the amino acids in the C-terminal region of the FGF1 in the amino acid sequences 152 to 155 represented by SEQ ID NOs. 1 to 5. In the mutant, another amino acid sequence may be inserted in the middle of the amino acid in the C-terminal region of this FGF1. As a typical example, an FGF1 mutant can be listed. In the FGF1 mutant, between the amino acid sequences 150 and 151 represented by SEQ ID NOs. 1 to 5, for example, the amino acid sequence derived from another origin, such as the CPP, is inserted, thus separating the C-terminal region. Meanwhile, with respect to the amino acid sequences of 1 to 150 represented by any of SEQ ID NOs. 1 to 5, the amino acid sequence preferably has the sequence identity of 90% or more and more preferably has the sequence identity of 95% or more.

For example, Non-Patent Document 9, 10, and 19 report the substitution and removal of some amino acids in the FGF1. This description incorporates these contents by reference.

### 1-2. FGF2

The FGF2 is also a physiologically active substance known for mammals, such as humans, mice, rats, cattle, and horses. The human FGF2 may have an amino acid sequence represented by SEQ ID NO. 6. The mouse FGF2 may have an amino acid sequence represented by SEQ ID NO. 7. The rat FGF2 may have an amino acid sequence represented by SEQ ID NO. 8. The cattle FGF2 may have an amino acid sequence represented by SEQ ID NO. 9. The horse FGF2 may have an amino acid sequence represented by SEQ ID NO. 10. The present invention may constitute the chimeric protein with the FGF2 derived from any mammal. For example, the FGF2 can be selected according to an animal to be treated.

Comparing the amino acid sequences of the FGF2s among these animals, the sequence not observed in the other animals is present at the N-terminal of the human FGF2. Meanwhile, a region having significantly high sequence identity, 95% to 99% is present among the all animals. This common domain is thought to pertain to the activity of the FGF2.

Specifically, the amino acids 134 to 288 in the amino acid sequence represented by SEQ ID NO. 6 and the amino acid sequences represented by SEQ ID NOs. 7 to 10 mutually have the sequence identity of 95% or more. With respect to any of these amino acid sequences, as long as the protein contains the amino acid sequence with the sequence identity of 80% or more, preferably 90% or more, and more preferably 95% or more, even if some amino acids in the amino acid sequence represented by any of SEQ ID NOs. 6 to 10 are substituted or deleted, or another amino acid is added, the FGF2 activity is thought to be provided.

Similar to the FGF1, it is thought that even if the mutant lacks all of or a part of the C-terminal region of the complete FGF2 or the mutant is the one where another amino acid sequence is inserted in the middle of the identical region and therefore the identical region is separated, the activity of the FGF2 is maintained. For example, the mutant may lack all of or some of the amino acids 283 to 288 in the amino acid sequence represented by SEQ ID NO. 6, the amino acids 149 to 154 in the amino acid sequences represented by SEQ ID NOs. 7 to 9, or the amino acids 150 to 155 in the amino acid sequence represented by SEQ ID NO. 10. In the mutant, another amino acid sequence may be inserted in the middle of the amino acid in the C-terminal region of this FGF1. As a typical example, an FGF2 mutant can be listed. In the FGF2 mutant, between the 282 and 283 in the amino acid sequence represented by SEQ ID NO. 6, between the 148 and 149 in the amino acid sequences represented by SEQ ID NOs. 7 to 9, or between the 149 and 150 in the amino acid sequence represented by SEQ ID NO. 10, for example, the amino acid sequence derived from another origin, such as the CPP, is inserted, thus separating the C-terminal region.

Comparing the domains common to these FGF2s with the FGF1 (the amino acids 1 to 155 in the SEQ ID NOs. 1 to 5), the domains have the sequence identity of 53% to 55%.

### 1-3. CPP

The chimeric protein used for the present invention as the active ingredient has a structure of fusing the CPP containing the FGF11 subfamily CPP-C domain (CPP-C) to the FGF1 or the FGF2. As the chimeric protein formed by fusing the CPP to the FGF1 or a similar factor, a chimeric protein formed by fusing a diphtheria toxin A to the FGF1 has been known. However, it had been understood that even if this chimeric protein is administered to translocate the FGF1 into the cells, this only synthesizes the DNA, but the FGFR needs to participate in this for the division and proliferation of the cells (Non-Patent Documents 4 and 24). Meanwhile, with the chimeric protein formed by fusing the CPP-C to the FGF1, the FGF1 provided various bioactivities.

The CPP-C can be obtained from mammals, such as humans, mice, rats, cattle, and horses. The CPP-C can be appropriately selected according to a target to be administered and a purpose of use of the chimeric protein or a similar condition.

For example, the CPP-C domains of the human FGF11 to 14 are each represented by the amino acid sequences shown by SEQ ID NOs. 11, 12, 13, and 14. The CPP-C domains of the mouse FGF11 to 14 are each represented by the amino acid sequences represented by SEQ ID NOs. 15, 16, 17, and 18. The CPP-C domains of the rat FGF11 to 14 are each represented by the amino acid sequences shown by SEQ ID NOs. 19, 20, 21, and 22. The CPP-C domains of the cattle FGF11 to 14 are each represented by the amino acid sequences shown by SEQ ID NOs. 23, 24, 25, and 26. The CPP-C domains of the horse FGF11, FGF13, and 14 are each represented by the amino acid sequences shown by SEQ ID NOs. 27, 28, and 29.

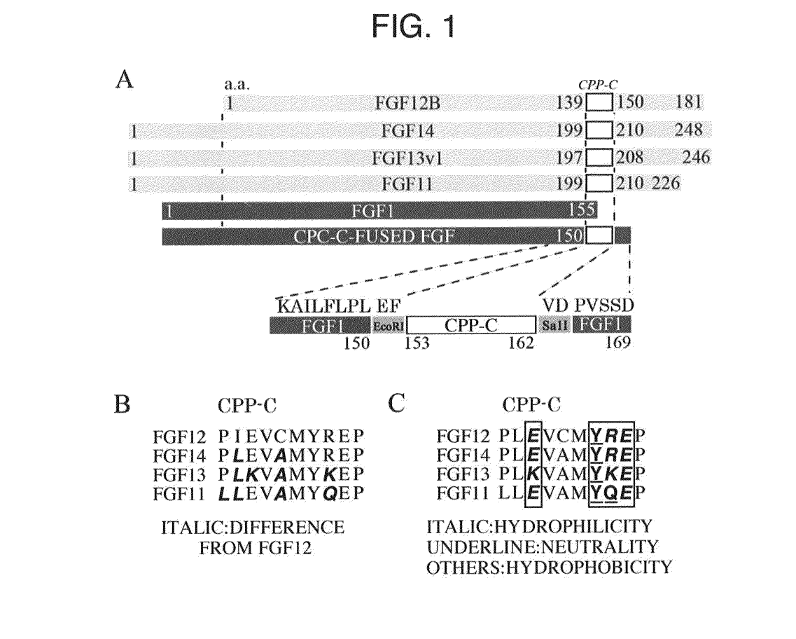

The sequence identity among the FGF11 subfamily CPP-C domains for animals is 80 to 100% with the FGF11, 100% with the FGF12, 100% with the FGF13, and 100% with the FGF14. For example, differences in the sequence among the FGF11 subfamily CPP-C domains for the human are as illustrated in FIG. 1B. The sequences have the sequence identity of 60% to 80% where 2 to 4 amino acids are mutually different. Meanwhile, as illustrated in FIG. 1C, for example, among the FGF11 subfamilies for the human, the sequence patterns of the hydrophilic amino acids or the neutral amino acids and the hydrophobic amino acids are common. The third and the ninth amino acids from the N-terminal side of the amino acid sequence constituting the CPP-C domain are hydrophilic, the seventh amino acids are neutral, the eighth amino acids are hydrophilic or neutral, and the other sites are all hydrophobic.

Therefore, as long as the mutant meets the following, the cellular internalization of the chimeric protein is considered to be possible. The sequence patterns of the CPP-C domain represented by any of the SEQ ID NOs. 11 to 29 and the hydrophilic amino acids or the neutral amino acids and the hydrophobic amino acids, and preferably the sequence patterns of the hydrophilic amino acids, the neutral amino acids, and the hydrophobic amino acids are common. Furthermore, the mutant belongs to the FGF11 subfamily CPP-C having the sequence identity of 60% or more, preferably 80% or more, and more preferably 90% or more. However, substitution among the amino acids with more similar polarity is preferable. For example, it is preferable that the peptide containing the CPP-C domain consisted of the following amino acids.

First: proline or leucine (preferably the proline)
Second: isoleucine or leucine (preferably the leucine)
Third: glutamic acid or lysine (preferably the glutamic acid)
Fourth: valine
Fifth: cysteine or alanine (preferably the alanine)
Sixth: methionine or valine (preferably the methionine)
Seventh: tyrosine
Eighth: arginine, lysine, or glutamine (preferably the arginine)
Ninth: glutamic acid
Tenth: proline

One or more amino acids may be further added to both or one of the terminals of the amino acid sequences constituting the CPP-C domain of the CPP, which constitutes the chimeric protein. For example, the CPP-C can be constituted of the amino acids of more than 10 to 40 or less. Alternatively, for example, the entire CPP-C can be derived from any of the FGF11 to 14 for various mammals, and the CPP-C can be constituted of the consecutive amino acids of more than 10. However, the less the additional amino acids is, the more a cell membrane permeation effect is. Therefore, the CPP-C containing the additional amino acids is preferably constituted of 40 or less amino acid residues, more preferably 25 or less, further preferably 20 or less, and more further preferably 15 or less, and is especially preferably constituted of only the CPP-C domains. From the similar aspect, in the case where the entire CPP-C is derived from the human FGF11 to 14, the CPP-C contains the amino acid sequence expressed by any of SEQ ID NOs. 11 to 14. Additionally, the CPP-C is preferably constituted of the consecutive amino acids of 40 or less, more preferably the consecutive amino acids of 25 or less, further preferably the consecutive amino acids of 20 or less, and more further preferably the consecutive amino acids of 15 or less. Especially preferably, the CPP-C is constituted of only the amino acid sequence represented by any of SEQ ID NOs. 11 to 14. Surely, with the amino acid sequence constituting the CPP, some of, preferably, within a several number of amino acids may be substituted while maintaining the above-described sequence patterns of the hydrophobic amino acids or the neutral amino acids and the hydrophilic amino acids in the CPP-C domain.

Non-Patent Document 8 describes the details of the CPP derived from the FGF11 subfamily member. This description incorporates the content by reference.

### 1-4. Joining of CPP to FGF1

The chimeric protein according to the present invention is formed by fusing the CPP-C to the FGF1 or FGF2. However, both may be directly joined and may be joined via a joining segment made of the peptide. The joining segment made of the peptide is preferably constituted of the hydrophilic amino acids such as the aspartic acid and the glutamic acid. From the point of stereostructure, the joining segment is preferably made of amino acids of less than 10, which is more preferable than that made of amino acids of less than 3.

In the case where another peptide is not joined, the CPP-C can be joined to the N-terminal side of the FGF1. However, usually, the CPP-C is joined to the C-terminal side or is inserted into the middle of the amino acid sequence in the C-terminal region. More specifically, for example, the CPP can be joined via the joining segment or without via the joining segment to the FGF1 mutant obtained by cutting the C-terminal side at any given position of the 151 to 155 in the amino acid sequences shown by SEQ ID NOs. 1 to 5, or to the C-terminal of the FGF1 mutant where the complete FGF1 or the C-terminal region is completely maintained. Alternatively, for example, the CPP-C can be inserted into any given position of the 151 to 155 in the amino acid sequences shown by SEQ ID NOs. 1 to 5 via one or two of the joining segments or without via the joining segment.

Similarly, the CPP can be joined via the joining segment or without via the joining segment to the FGF2 mutant obtained by deleting the C-terminal side at any given position of the amino acids 283 to 288 in the amino acid sequence represented by SEQ ID NO. 6, the amino acids 149 to 154 in the amino acid sequences represented by SEQ ID NOs. 7 to 9, or the amino acids 150 to 155 in the amino acid sequence represented by SEQ ID NO. 10, or to the C-terminal of the FGF2 mutant where the complete FGF2 or the C-terminal region is completely maintained. Alternatively, for example, the CPP-C can be inserted into any given position of the amino acids 283 to 288 in the amino acid sequence represented by SEQ ID NO. 6, the amino acids 149 to 154 in the amino acid sequences represented by SEQ ID NOs. 7 to 9, or the amino acids 150 to 155 in the amino acid sequence represented by SEQ ID NO. 10 via one or two of the joining segments or without via the joining segment.

Such constitution allows introducing the CPP-C while designing the amino acid sequence featuring high homology with the amino acid sequence of the original FGF1 or FGF2. Therefore, this constitution is preferable from the point of maintaining the original functions of the FGF1 or FGF2.

FIG. 1A schematically illustrates the structure of the CPP-FGF1 chimeric protein according to a preferred embodiment of the present invention. This embodiment separates the amino acid sequence of the FGF1 between 150 and 151. The CPP-C of the FGF11 subfamily member is inserted into the position via an EcoRI cleavage sequence and a SalI cleavage sequence. In this embodiment, the CPP-C is constituted only with 10 residues of amino acids constituting the CPP-C domain. The 1 to 150 in the amino acid sequence of the FGF1 are maintained. Therefore, it is thought that the CPP-C features high cell membrane permeability, and the biological or pharmacological activity of the FGF1 is completely maintained. Actually, as proved in the working example, which will be described later, the CPP-C can provide various high-level pharmacological actions. The specific amino acid sequences of such chimeric protein are shown by SEQ ID NOs. 30 to 33.

### 1-5. Method for Preparing Chimeric Protein

The following describes an exemplary method for preparing the above-described CPP-FGF1 or CPP-FGF2 chimeric protein.

The DNA coding the FGF1 or FGF2 is replicated by synthesis, polymerase chain reaction (PCR), or a similar method. A restriction enzyme cleavage site is added to an appropriate site of this DNA and is cleaved by restriction enzyme.

Meanwhile, the CPP is coded, a single-stranded DNA fragment also having the corresponding restriction enzyme cleavage terminal is synthesized, and a double-stranded DNA fragment is formed by annealing. Afterwards, using a DNA ligase, the DNA fragment coding the CPP is inserted into and joined to the cleavage site of the DNA that codes the FGF1 or FGF2. One kind or two kinds of the restriction enzymes can be used.

As vectors that incorporate the DNA, which codes this chimeric protein, as long as the vectors are reproduced and held in a host, any vectors can be used. For example, the vector is an expression vector, such as a plasmid from Escherichia coli (pBR322, pBR325, pUC12, and pET-3), a plasmid from Bacillus subtilis, a bacteriophage such as a gamma phage, a derivative of the bacteriophage, an animal virus such as a retrovirus, an adenovirus, and a vaccinia virus, and an insect virus.

The gene of the chimeric protein may have an ATG as a translation initiation codon at the 5′-terminal. The gene of the chimeric protein may have a TAA, TGA, or the ATG as a translation termination codon at the 3′-terminal. These expression vectors preferably contain a promotor at upstream of the code sequence of the CPP-FGF chimeric protein. The gene can be preferably expressed on the host. Insofar as the promotor is appropriate for the host used for the expression of the gene, any promotor may be used.

As the host, for example, the Escherichia coli (for example, BL21, BL21 (DE3), BL21 (DE3) pLysS, and BL21 (DE3) pLysE), the Bacillus subtilis (for example, Bacillus subtilis DB305), the yeast (for example, Pichia pastoris and Saccharomyces cerevisiae), the animal cell (for example, a COS cell, a CHO cell, a BHK cell, an NIH3T2 cell, a I-TUVE cell, and a LEII cell), and the insect cell are listed.

For transformation, it is only necessary to select an applicable method according to each host. For example, in the case where the host is the Escherichia coli, a heat shock method and an electroporation method can introduce recombinant DNAs or the vectors into competent cells made by a calcium method or another method.

Thus, a transformant that holds the vectors containing the recombinant DNAs coding the CPP-FGF1 chimeric protein is obtained. Culturing this transformant produces the CPP-FGF1 chimeric protein. It is only necessary to select a culture medium appropriate to the culture of the transformant according to the host. For example, in the case where the host is the Escherichia coli, an LB culture medium is used. In the case where the host is the yeast, an YPD culture medium or a similar culture medium is used. It is also only necessary to accordingly select a culture condition appropriately according to each host. For example, in the case where the host is the Escherichia coli, the culture is performed at around 30 to 37° C. for around 3 to 24 hours, and as necessary, ventilation and stir can be added.

After the culture, as a method for breaking cultured bacterial cells or the cultured cells to elute the chimeric protein, for example, a homogenizer, a French press, an ultrasonic wave, a lysozyme, and a freeze thaw are listed. The chimeric protein can be purified by one of or in combination with the known separation method from a soluble fraction and a purification method. Such separation method or purification method can include, for example, a salting-out, a solvent precipitation, a dialysis, an ultrafiltration, a gel filtration, an SDS-polyacrylamide gel electrophoresis, an ion exchange chromatography, an affinity chromatography, a reversed-phase high-performance liquid chromatography, and an isoelectric-focusing electrophoresis. As one preferable example, in the case where a heparin-binding domain is saved in the FGF1 part of the chimeric protein, a method that uses the heparin binding to isolate the chimeric protein can be listed. Specifically, for example, the heparin-sepharose chromatography is caused to adsorb the chimeric protein, and the chimeric protein is eluted using a gradient of sodium chloride. Thus, the chimeric protein is separated and purified.

Usually, the chimeric protein obtained as described above is preferably refrigerated at 4° C. or less or is kept in a freezer. As long as the activity is not lost, dialyzing the chimeric protein to substitute the chimeric protein with an appropriate solvent is also possible. Furthermore, freeze drying can also be performed to form the chimeric protein into dry powder.

### 2. Recombinant DNAs or Vectors

The present invention can use the recombinant DNA that codes the above-described chimeric protein or the vector that includes such recombinant DNA as the active ingredient. This embodiment, for example, can express the above-described chimeric protein inside the body using such recombinant DNAs or vectors for target treatment.

As the recombinant DNA, the following can be listed as a typical example. The recombinant DNA includes a DNA sequence having a sequence identity of at least 60%, preferably 70% or more, more preferably 80% or more, and especially preferably 90% or more with respect to a DNA sequence coding the amino acid sequence of the FGF1 represented by any of SEQ ID NOs. 1 to 5 or the FGF2 represented by any of SEQ ID NOs. 6 to 10. The recombinant DNA also includes a DNA sequence coding an amino acid sequence having the identical sequence pattern to the sequence pattern of the FGF11 subfamily CPP-C domain represented by any of SEQ ID NOs. 11 to 29 or the FGF11 subfamily CPP-C domain and the hydrophobic amino acids or the neutral amino acids and the hydrophilic amino acids.

As the vectors, the vectors generally used for a gene therapy may be used, for example, an adenovirus, a retrovirus, a hemagglutinating virus of Japan, and a plasmid. Preferable vectors can be selected according to the object. Especially, the hemagglutinating virus of Japan is preferable.

The method for introducing and expressing a chimeric DNA of the present invention in a living body includes, for example, a membrane fusion liposome and nanoparticles.

### 3. Medicinal Use of Chimeric Protein

The CPP-FGF1 chimeric protein of the present invention contains the FGF1 as the main component part. Accordingly, the CPP-FGF1 chimeric protein is effective to symptoms or diseases that can be prevented or treated by the native FGF1. Therefore, not only in the stage of development also for the adult, the CPP-FGF1 chimeric protein is effective to various medical uses involving cell divisions at various tissues, such as the brain, the central nerve, the kidney, the placenta, the adrenal gland, the skin, the hair, the eardrum, the eye, and the digestive tract such as the intestinal tract; and a physiological action, such as a cell proliferation, an anti-apoptosis, protection of stem cells, and vascularization. For example, the chimeric protein of the present invention is not limited to these. The chimeric protein is effective for prevention or treatment of an exfoliation, a degeneration, a ulcer, a necrosis, a damage, or a damage of a tissue, such as the brain, the central nerve, the kidney, the placenta, the adrenal gland, the skin, the hair, the eardrum, the eye, the digestive tract such as the intestinal tract, and the reproductive tissue such as the ovary caused by radiation, chemotherapy, physical intervention, apoptosis, or other causes. Alternatively, the chimeric protein is effective for prevention or treatment of an ischemic symptom or a disease such as a limb ischemia or an ischemic coronary artery disease; or a proliferation, a metastasis, or a similar phenomenon of the lung cancer, the stomach cancer, the colon cancer, the pancreatic cancer, the renal cell carcinoma, the squamous cell carcinoma, the malignant melanoma, the uterine cancer, the ovarian cancer, the bladder cancer, the ureter cancer, and the tumor cell such as the angiosarcoma.

The CPP-FGF1 or CPP-FGF2 chimeric protein according to the present invention is translocated into the cells independent of the FGFR, allowing activating the biological or pharmacological activity by the FGF1 or the FGF2. In view of this, the chimeric protein of the present invention is especially effective for the prevention or treatment of the hematopoietic cells such as the lymphocyte where the expression of the FGFR is originally low, the tissue where the expression of the FGFR is deteriorated due to various factors, such as a burn, radiation, a deficiency of blood supply, and an infection, a tumor where a profile for the expression of the FGFR differs from a normal tissue, or a symptom where the FGF1 or the FGF2 cannot be translocated into the cells or cannot interact with the FGFR due to any cause. Under these symptoms or diseases, the low expression of the FGFR or a similar factor becomes an obstacle; therefore, the administration of the native FGF1 or a similar factor failed to obtain sufficient prevention or curative effect. Accordingly, the composition of the present invention can bring improved prevention and curative effects for these symptoms or diseases. Examples of these diseases or symptoms are: the prevention, the treatment, or a similar medical practice of a disorder of a skin tissue by burn, a disorder of a tissue such as the intestinal tract induced by radiation or chemotherapy, an exfoliation of a tissue caused by an apoptosis induced by, for example, radiation, such as an alopecia induced by radiation or chemotherapy, an ischemic symptom or a disease such as a limb ischemia or an ischemic coronary artery disease, a diabetic skin ulcer or a diabetic gangrene, the proliferation, the metastasis, or a similar phenomenon of the lung cancer, the stomach cancer, the colon cancer, the pancreatic cancer, the renal cell carcinoma, the squamous cell carcinoma, the malignant melanoma, the uterine cancer, the ovarian cancer, the bladder cancer, the ureter cancer, and the tumor cell such as the angiosarcoma.

Other constituents of the medicinal composition containing the chimeric protein or a similar component according to the present invention are not especially limited. For example, using a medicinally allowable solvent, such as a diluent, an excipient, a carrier, and an adjuvant, in accordance with a common procedure for formulation and production, the medicinal composition can be prepared to dosage forms, such as a liquid medicine, a parenteral injection, powder, a granule, a tablet, a suppository, an ointment, an enteric tablet, or a capsule. The medicinal composition according to the present invention is not especially limited regarding an administration route as well. According to an indication, dosage forms, or a similar condition, the medicinal composition can be administered orally or parenterally, such as intravascularly, subcutaneously, intraperitoneally, and intratumorally. A dose of the medicinal composition according to the present invention is appropriately changed depending on the dosage forms, the administration route, and the symptom. For example, to intravenously administrate the medicinal composition to the mammal including the human, administrating the chimeric protein of about 0.001 to 1 mg/weight kg for one day is preferable. For administration by being injected subcutaneously, administrating the chimeric protein of about 0.01 to 10 mg/weight kg for one day is preferable.

The medicinal composition of the present invention may contain an active ingredient as well as the CPP-FGF1 chimeric protein. The additional active ingredient can include, for example, cytokine such as G-CSF, other cell proliferation factors such as VEGF, HGF, and EGF, or a molecularly targeted drug targeting these factors.

The active ingredient thus used together is selected according to an indication. For example, in the case of treating the tumor, the molecularly targeted drug or a similar drug can be combined. For prevention or treatment of a radiation disorder, the cytokine, the growth factor, or a similar active ingredient can be combined.

### Working Examples

Hereafter, the present invention is described in greater detail using the working examples, although the technical scope of the present invention is not limited to these working examples.

### Testing Methods and Testing Materials

The following summarizes testing methods and materials used for each working example.

### 1. FGF1, FGF12B, and FGF12B Fragments

The FGF1 having the amino acid sequence shown by SEQ ID NO. 1 was prepared in accordance with the method described in Non-Patent Document 8. The FGF12B and FGF12B fragments were also prepared in the procedure described in Non-Patent Document 8. The amino acid sequence of the FGF12B is shown by SEQ ID NO. 34.

### 2. Chimeric Protein

The chimeric proteins formed by fusing each CPP-C derived from the FGF11, FGF12, FGF13, and FGF14 of the FGF11 subfamily to the FGF1 (hereinafter abbreviated as a CPPF1, CPPF2, CPPF3, and CPPF4, respectively) were prepared in accordance with the method described in Non-Patent Document 8. The related description of Non-Patent Document 8 is incorporated here by reference.

FIG. 1A illustrates the structure of each chimeric protein. The amino acid sequences of the chimeric proteins are shown by SEQ ID NOs. 30 to 33. 3. FACS

In accordance with the method described in Non-Patent Document 8, each FGF was fluorescence-labeled. The fluorescence intensity was measured with FACS Calibur (manufactured by BD Biosciences).

### 4. TUNEL Assay

In accordance with the method described in Non-Patent Document 8, an apoptosis was detected from a paraffin-embedded section of a mouse tissue.

### 5. Experimental Mouse

Treatments of mice in each working example were conducted based on animal ethics described in the Animal Protocol, which had been preliminary approved by the Institutional Animal Care and Use Committee of the National Institute of Radiological Sciences.

### Evaluation for Cellular Internalization Ability 1

This test evaluated the cellular internalization ability of the CPP-FGF1 chimeric protein on the cells where the expression of the FGFR was low.

This test used rat small intestinal cell lines TEC6 as testing cells. The testing cells were seeded on a 24-well plate by 1×10⁵ cells for each well. A DMEM culture medium containing 5% FCS and 4 μg/ml insulin was added to each well and was cultured for six hours. The cells were accumulated to the plate. Afterwards, the fluorescence-labeled FGF12B, each fluorescence-labeled FGF12B fragment (Δ170 to 181, Δ160 to 181, Δ150 to 181, and Δ140 to 181) from which the C-terminals were additionally removed in units of ten residues, fluorescence-labeled FGF1, CPPF1, CPPF2, CPPF3, and CPPF4 were added to the plate so as to be each 1 μg/ml. After the culture for 24 hours, the cells were harvested from the plate with trypsin. The fluorescence intensity of the cells was measured by the FACS, and the amount of the FGF translocated into the cells was measured.

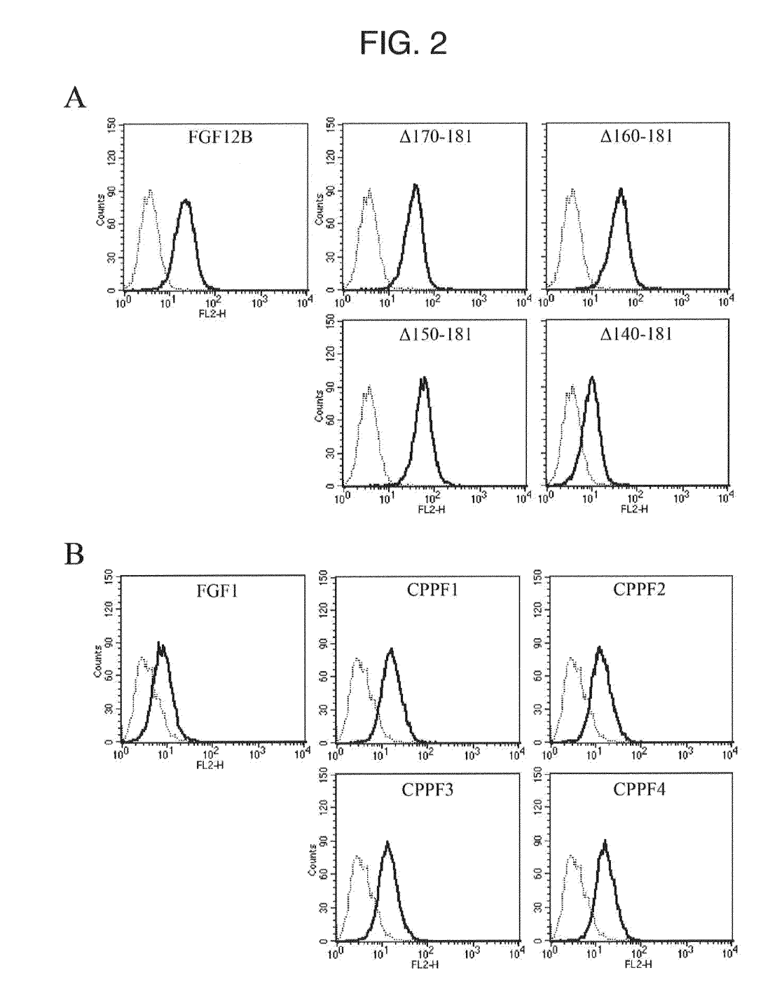

FIG. 2A illustrates FACS histograms before and after addition of the FGF12B or each FGF12B fragment. FIG. 2B illustrates FACS histograms before and after addition of the FGF1 or each CPP-FGF1 chimeric protein. The dotted lines indicate the FACS histograms for the cells before addition of each FGF. The solid lines indicate the FACS histograms for the cells after addition of each FGF.

As illustrated in FIG. 2A, with the cell cultured by the FGF12B, the cell population indicated by the solid line greatly moves rightward. Accordingly, it can be seen that the fluorescence intensity of the cell is intensive. Even if the residues were lost in units of ten from the C-terminal of the FGF12B, with the FGF12B fragments maintaining the amino acid residues 1 to 149 (Δ170-181, Δ160-181, and Δ150-181), the fluorescence intensity hardly changed and remained intensive. However, among these fragments, the fluorescence intensity was the maximum at the fragment where the shortest amino acid residues 150-181 were removed. Meanwhile, with the fragment where the amino acid residues 140-181 were removed, the fluorescence intensity rapidly weakened. From these results, it is presumed that the amino acid residues 140-149 of the FGF12B were the CPP-C domain. Meanwhile, it has been shown that even if the amino acids are added to before and after this CPP-C domain, the cellular internalization ability is acquired. It has also been guessed that less number of amino acids added to before and after the CPP-C domain provides higher cellular internalization ability.

As illustrated in FIG. 2B, with the cells cultured with the FGF1, the cell population indicated by the solid line moves rightward little. Accordingly, it can be seen that the fluorescence intensity of the cell is weak. Meanwhile, with the cells cultured together with each chimeric protein (the CPPF1, CPPF2, CPPF3, and CPPF4), the cell population indicated by the solid line greatly moves rightward compared with the FGF1. Accordingly, it can be seen that the fluorescence intensity of the cells are intensive. Among the four kinds of chimeric proteins, the fluorescence intensity was almost identical. From this result, it has been proved that, compared with the FGF1, the CPP-FGF1 chimeric proteins were able to be translocated into the cells efficiently.

### Evaluation for Inhibitory Effect on Apoptosis 1

This test evaluated the inhibitory effect of CPP-FGF1 chimeric protein on radiation-induced apoptosis in the cells.

This test also used the rat small intestinal cell lines IEC6 not expressing an FGFR as testing cells. The cells were plated at 3×10⁴ cells per 3.5-cm dish. The DMEM culture medium containing 5% FCS and 4 μg/ml insulin was added to each dish. Each dish was put into an incubator under an atmosphere at 37° C. and 5% CO₂ and then was cultured for 16 hours. Next, the heparin at a concentration of 5 μg/ml was added to each culture medium. In a control group, the FGF was not added. In each experimental group, the FGF1, CPPF1, CPPF2, CPPF3, and CPPF4 were added at a concentration of 100 ng/ml, respectively. Further, after the culture for 24 hours, X-rays were irradiated at 20 Gy. After the irradiation for 24 hours, the cells were fixed with 2% glutaraldehyde, and nuclear staining was performed with 20 μg/ml Hoechst 33258. Then, one visual field with the cells of 200 or more was examined microscopically by ten visual fields with an inverted fluorescence microscope. Thus, the number of cells involving the pycnosis was calculated. This pyknotic cells were determined as cells that induced the apoptosis by irradiation with X-rays. A proportion of the number of pyknotic cells to a total number of the cells microscopically examined in each visual field was evaluated as the apoptosis proportion.

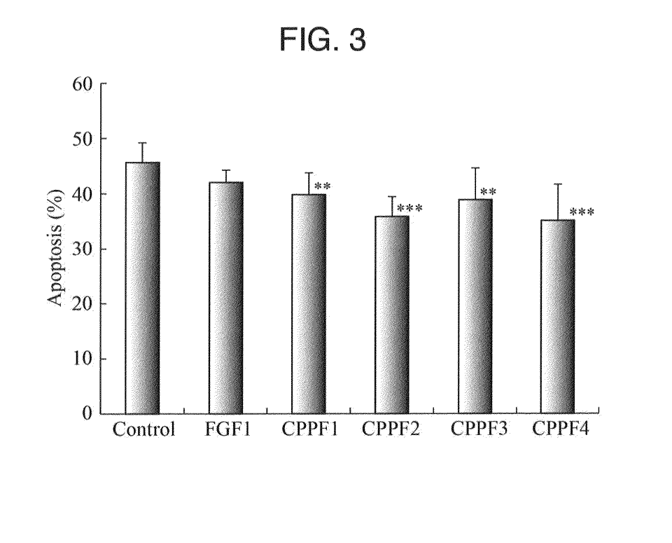

FIG. 3 shows an average value+/− standard deviation (S.D.) of the apoptosis proportion of the control group and each experimental group. In the drawing, ** indicates the experimental groups where P<0.01 was met by a multiple test on the control group. *** indicates the experimental groups where P<0.001 was met by the identical test.

In the control group that did not contain the FGF, the apoptosis proportion reached to about 45%. Even in the experimental group with addition of the FGF1, a significant reduction in apoptosis proportion was not observed compared with the control group. Meanwhile, in the experimental groups with addition of the CPP-FGF1 chimeric proteins (CPPF1, CPPF2, CPPF3, and CPPF4), all the apoptosis proportions were significantly reduced compared with the control group. This has proved the following. The FGF1 fails to effectively inhibit the apoptosis of cells expressing no FGFR. Meanwhile, the CPP-FGF1 chimeric protein can inhibit the apoptosis even on such cells. The fact that the FGF1, which is originally expected to inhibit the apoptosis via the FGFR, did not exhibit a significant difference from the control is consistent to the fact that the expression of the FGFR on the IEC6 cell was not confirmed. Meanwhile, the fact that the CPP-FGF1 chimeric protein can inhibit the apoptosis even under such condition, it is highly possible that this situation is caused by a property of the CPP-FGF1 chimeric protein being able to be translocated into the cells independent of the expression of the FGFR.

### Evaluation for Inhibitory Effect on Apoptosis 2

This test evaluated the inhibitory effect of FGF12B and FGF12 fragments on radiation-induced apoptosis in the cells.

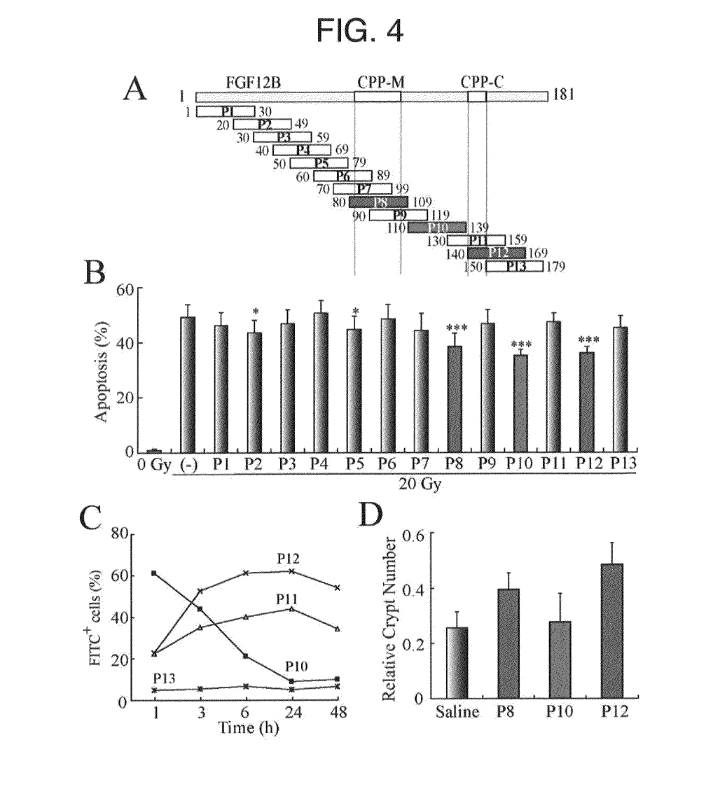

This test used the FGF12B and each FGF12 fragments illustrated in FIG. 4A as the FGFs. The P8 fragment contains the CPP-M, and the P11 and P12 fragments contain the CPP-C. As the testing cells, this test also used the rat small intestinal cell line IEC6.

The testing procedure is similar to the evaluation regarding the above-described inhibitory effect on the apoptosis. FIG. 4B shows an average value +/− standard deviation (S.D.) of the apoptosis proportion of the control group and each experimental group. In the drawing, * indicates the experimental groups where P<0.05 was met by the multiple test on the control group. *** indicates the experimental groups where P<0.001 was met by the identical test.

In the control group that did not contain the peptide, the apoptosis proportion reached to about 45%. In all the experimental groups with the addition of the P8, P10, and P12, the apoptosis proportion was significantly reduced compared with the control group. Meanwhile, after all, in the experimental group with the addition of the P11, which contained the CPP-C, the apoptosis proportion was not significantly reduced compared with the control group. This has proved that the P12 made of the 30 amino acids containing the CPP-C reduces the apoptosis, while the CPP-C itself made of the 10 amino acids cannot inhibit the apoptosis. Furthermore, it has also been proved that the peptide containing the CPP-M domain of the center reduces the apoptosis.

FIG. 4C shows the cellular internalization ability of the C-terminal peptide of the FGF12B. FIG. 4C is a graph where a positive fluorescence rate of the IEC6 cell line is measured over time by the FACS after addition of each fluorescence-labeled peptide at the concentration of 10 μg/ml. Peaking at 24 hours, the P12, which contained the CPP-C, was translocated into the cells. Although the P11, which similarly contained the CPP-C, exhibited the positive fluorescence rate lower than the P12, similar to the P12, the P11 was translocated into the cells peaking at 24 hours. Meanwhile, the P10 and the P13 exhibited extremely low positive fluorescence rate after the elapse of 24 hours.

FIG. 4D is a graph showing average values of a crypt survival rate in a peptide or a saline intraperitoneal administration group. Eight-week old male BALB/c mice were used. In the control group, a 0.5 ml of saline containing 5% mouse serum was administered to the abdominal cavity of the mouse. In the experimental groups, each 100 μg of the P8, P10, and P12 was diluted with the 0.5 ml of saline containing 5% mouse serum and was administered to the abdominal cavity of the mouse. After the elapse of 24 hours, the gamma rays at 10 Gy were irradiated at a dose rate of 0.5 Gy/min on the whole body of the mice from each group. After the elapse of three and half days from the irradiation, the mice were euthanized and then the jejuna were sampled. After fixing the jejuna with 10% formalin, the paraffin-embedded sections were made and the sections were stained with HE. Using the microscope, a crypt where ten or more of crypt cells were present was determined as survived. The number of crypts of each cross section was counted on ten cross sections of intestine, and the average number of crypts was calculated. Furthermore, this average value was divided by the average number of crypts in each cross section in a non-irradiated group to obtain a relative value (a crypt survival rate). The graph shows the average value +/− standard deviation (S.D.) of the crypt survival rate of three individuals of mice in each group.

The group administrating the P8 or the P12 exhibited significantly high crypt survival rate in the jejunum compared with the control group. However, the group administrating the P10 did not exhibit significantly high crypt survival rate in the jejunum compared with the control group.

### Evaluation for Preventive Effect Against Hair Follicle Damage

This test evaluated the preventive effect of CPP-FGF1 chimeric protein against hair loss and hair follicle damages induced by radiation. In the hair follicles, the cells are divided actively in the growth phase during which the hair follicles are highly sensitive to radiation. In view of this, irradiation on the hair follicles in this period is likely to cause the apoptosis. This apoptosis indexes for the hair follicle disorder. Accordingly, an inhibitory effect of CPP-FGF1 chimeric protein on radiation-induced apoptosis was measured in the hair follicles of the mice in the growth phase to evaluate the preventive effect against the hair follicle damage.

Backs of male BALB/c mice of 51 to 53 days old after birth were depilated. Thus, the hair follicles in the resting stage were induced to the growth phase. At the elapse of five days after the depilation, in the control group, 0.5 ml of saline containing 5% mouse serum was administered to the abdominal cavity of the mouse. In the experimental groups, each 100 μg of FGF1, FGF12, CPPF1, CPPF2, CPPF3, and CPPF4 was diluted with the 0.5 ml of saline containing 5% mouse serum and was administered to the abdominal cavity of the mouse. After the elapse of 24 hours, the gamma rays at 12 Gy were irradiated at the dose rate of 0.5 Gy/min on the whole body. After the elapse of 24 hours from the irradiation, the mice were euthanized and then the skins were sampled. Afterwards, the skins were fixed with 10% formalin, the paraffin-embedded sections were made, and the TUNEL assay was performed. The TUNEL positive cells were regarded as apoptotic cells, and the number of apoptosis of each hair follicle bulb was calculated at three or more visual fields.

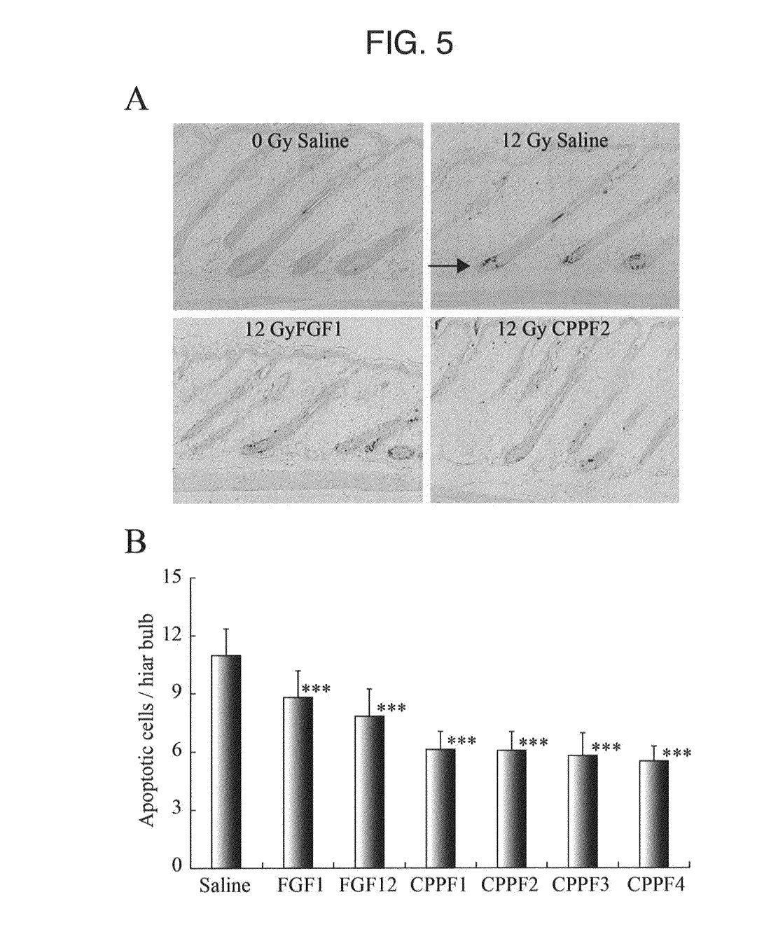

FIG. 5A includes photomicrographies (200 powers) of immunohistostaining on hair follicle bulb regions of mice from each group by the TUNEL assay. The arrow in the drawing indicates the TUNEL positive cell (namely, the apoptotic cell). FIG. 5B shows the average value+/− standard deviation (S.D.) of the number of apoptosis of each hair follicle bulb at three or more visual fields in each group. In the drawing, *** indicates the experimental groups meeting P<0.001 by the multiple test on the control group where the saline containing 5% mouse serum was administered.

In the control group, by total body irradiation with gamma rays at 12 Gy, about 11 cells of the TUNEL positive cells exhibiting apoptosis in the hair follicle bulb region were detected from each hair follicle bulb. In the FGF1 administration group and the FGF12 administration group, the number of TUNEL positive cells on each hair follicle bulb was significantly reduced compared with the control group. In the groups administrating the CPP-FGF1 chimeric proteins (CPPF1, CPPF2, CPPF3, and CPPF4), the number of all TUNEL positive cells on each hair follicle bulb significantly reduced compared with not only the control group but also the FGF1 administration group (P<0.001) and the FGF12 administration group (P<0.05). This has proved that the CPP-FGF1 chimeric protein exhibited the preventive effect against the hair loss and hair follicle disorders much higher than the FGF1 and the FGF12.

### Preventive Effect Against Radiation-Induced Intestinal Damage

This test evaluated the preventive effect of CPP-FGF1 chimeric proteins against radiation-induced intestinal damage. The crypts where the stem cells are present play a considerably important role in a recovery process of a small intestinal epithelium damaged by exposure to radiation. Therefore, the extent of radiation damage correlates the number of apoptosis present at the crypts. In view of this, the number of apoptosis at the crypt of the mouse, which was irradiated, was measured to evaluate the preventive effect of CPP-FGF1 against radiation-induced intestinal damage.

Eight-week old male BALB/c mice were used. In the control group, 0.5 ml of saline containing 5% mouse serum was administered to the abdominal cavity of the mouse. In the experimental groups, each 100μg of the FGF1, FGF12, CPPF1, CPPF2, CPPF3, and CPPF4 was diluted in 0.5 ml of saline containing 5% mouse serum and was administered intraperitoneally into the mouse. After the elapse of 24 hours, the gamma rays at 12 Gy were irradiated at the dose rate of 0.5 Gy/min on the whole body of each mouse. At 24 hours after irradiation, the mice were euthanized and then the small intestines were sampled. Afterwards, the small intestines were fixed with 10% formalin, the paraffin-embedded sections were made, and the TUNEL assay was performed. The TUNEL positive cells were regarded as apoptotic cells, and the number of apoptosis at each crypt was calculated at ten fields.

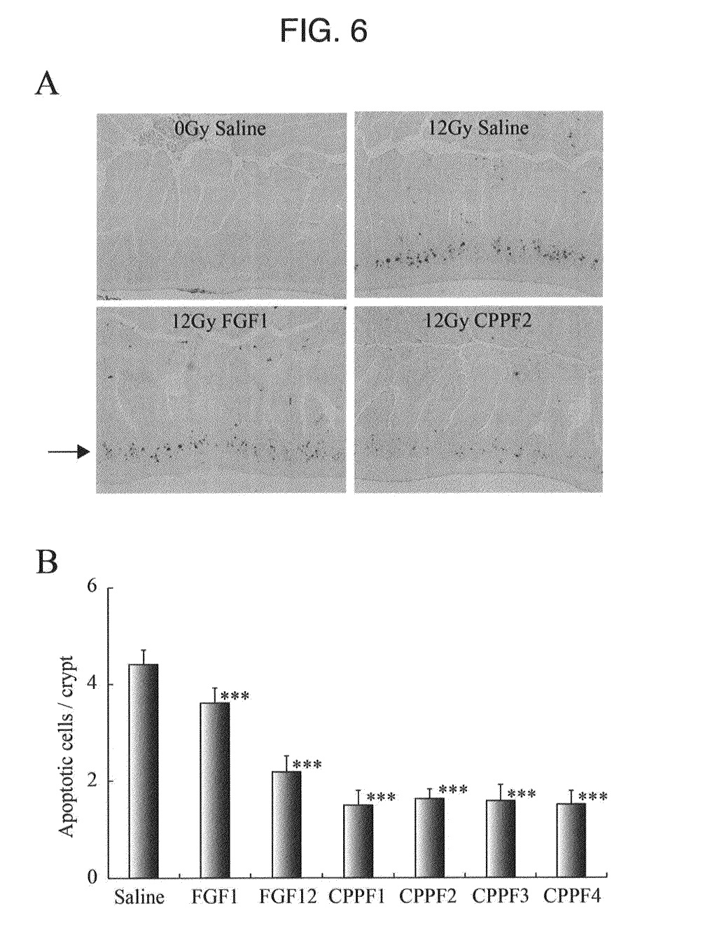

FIG. 6A includes photomicrographies when performing the immunohistostaining on crypts in the small intestines of mice from each group by the TUNEL assay. The arrow in the drawing indicates the TUNEL positive cell (namely, the apoptotic cell). FIG. 6B shows the average value+/− standard deviation (S.D.) of the number of TUNEL positive cells at each crypt in ten visual fields in each group. In the drawing, *** indicates the experimental groups meeting P<0.001 by the multiple test on the control group where the saline containing 5% mouse serum was administered.

As illustrated in FIG. 6B, in the control group, after total body irradiation with gamma rays at 12 Gy, the number of apoptosis was detected by 4.41 in average at each crypt. However, in the FGF1 administration group, the number of apoptosis was significantly reduced to 3.61 in average (P<0.001). In the FGF12 administration group, the number of apoptosis was also significantly reduced to 2.18 in average (P<0.001). In the groups administrating each CPP-FGF1 chimeric protein (CPPF1, CPPF2, CPPF3 and CPPF4), the number of apoptosis were remarkably reduced to 1.50, 1.63, 1.58, and 1.51 in average, respectively. An apoptosis reduction ratio of the FGF1 administration group compared with the control group was only 18.1%. Meanwhile, the apoptosis reduction ratio was 66% in the CPPF1 administration group, 63.1% in the CPPF2 administration group, 64.2% in the CPPF3 administration group, and 65.8% in the CPPF4 administration group, all of which reach 60% or more. These CPP-FGF1 administration groups significantly reduced the apoptosis (P<0.001) compared with the FGF1 administration group as well. The apoptosis reduction ratio of the FGF12 administration group compared with the control group was 50.5%, also significantly reducing the apoptosis compared with the FGF12 group.

This has proved that the CPP-FGF chimeric protein has high protective effect against radiation-induced intestinal damage compared with the FGF1 and the FGF12.

### Evaluation for Effect on Promoting Recovery of Damaged Small Intestine 1

This test indexed the number of crypts regenerated after irradiation to evaluate the effect of CPP-FGF1 chimeric protein on promoting the recovery of small intestine damaged by radiation.

Eight-week old male BALB/c mice were used. First, the gamma rays at 10 Gy were irradiated at a dose rate of 0.5 Gy/min on the whole body of the mice from each group. After the elapse of 24 hours from the irradiation, in the control group, a 0.5 ml of saline containing 5% mouse serum was administered to the abdominal cavity of the mouse. In the experimental groups, each 10 μg of the FGF1, CPPF1, CPPF2, CPPF3, and CPPF4 was diluted with the 0.5 ml of saline containing 5% mouse serum and was administered to the abdominal cavity of the mouse. After the elapse of three and half days from the irradiation, BrdU was incorporated into the dividing cells by intraperitoneal injection of a BrdU labeling solution. After the elapse of two hours, the mice were euthanized, and the jejuna were sampled. After fixing the jejuna with 10% formalin, the paraffin-embedded sections were made. The immunohistostaining was performed on this section with an anti-BrdU antibody, and then this section was stained with hematoxylin.

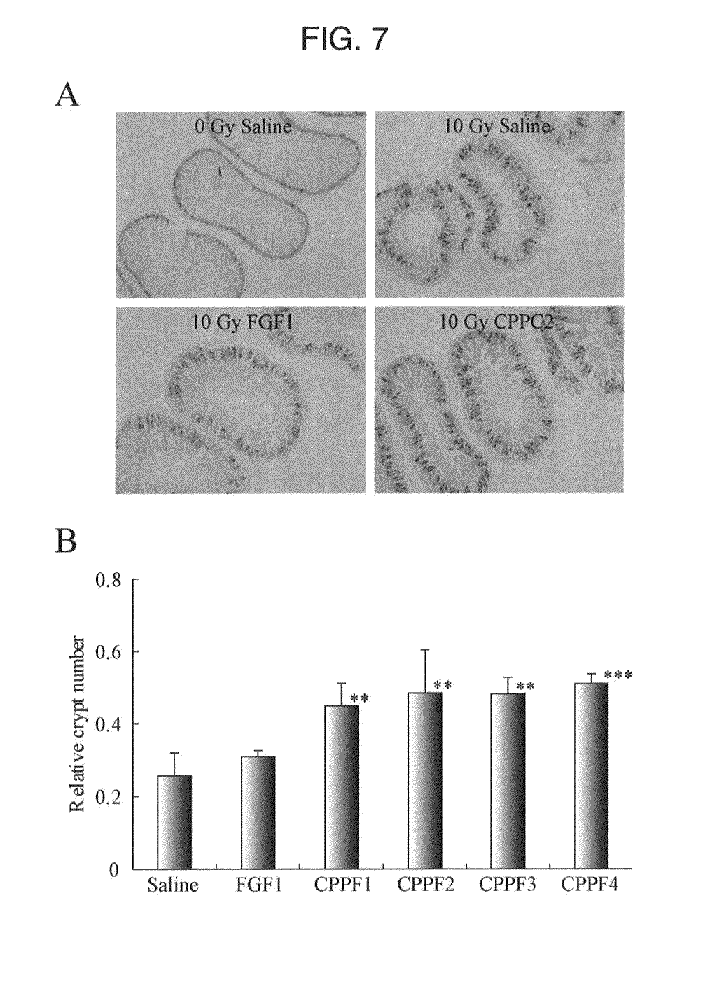

FIG. 7A includes photomicrographies of cross-sectional intestines that show crypts with cells incorporating BrdU and to which the anti-BrdU antibodies are bound. Using the microscope, a crypt where ten or more of anti-BrdU antibody positive cells were present was determined as survived. The number of crypts of each cross section was counted on ten cross sections of intestine, and the average value of the number of crypts was calculated. Furthermore, this average value was divided into the average value of the number of crypts in each cross section in a non-irradiated group to obtain the relative value (the crypt survival rate). FIG. 7B shows the average value+1− standard deviation (S.D.) of the crypt survival rate of three individuals of mice from each group. In the drawing, ** indicates the experimental groups meeting P<0.01 by the multiple test on the control group where the saline containing 5% mouse serum was administered while *** indicates the group meeting P<0.001 by the identical test.

In the control group administrating the saline containing 5% mouse serum, the crypt survival rate of the jejunum after irradiation of gamma rays at 10 Gy on the whole body was only 0.26. Even in the FGF1 administration group, the crypt survival rate was not significantly increased. Meanwhile, in the groups administrating each CPP-FGF1 chimeric protein (CPPF1, CPPF2, CPPF3 and CPPF4), the crypt survival rates of the jejunum were 0.45, 0.48, 0.48, and 0.51, respectively, being significantly high crypt survival rates compared with not only the control group but also the FGF1 administration group (P<0.05). This result also has proved that the CPP-FGF1 chimeric protein exhibited extremely high effect to promote the recovery of the small intestine damaged by radiation compared with the FGF1.

### Evaluation for Effect on Promoting Recovery of Damaged Small Intestine 2

This test also evaluated the effect of CPP-FGF1 chimeric protein on promoting the recovery from radiation-induced intestinal damage. However, this test indexes the length of the crypt. The length of the crypt reflects the number of cells present at the crypt, namely, the proliferation ability of the epithelial cell. Accordingly, the length becomes a good index for evaluation on recovery capability from the disorder of the small intestine.

Eight-week old male BALB/c mice were used. The gamma rays at 10 Gy were irradiated at a dose rate of 0.5 Gy/min on the whole body of the mice from each group. After the elapse of 24 hours from the irradiation, in the control group, 0.5 ml of saline containing 5% mouse serum was administered to the abdominal cavity of the mouse. In the experimental groups, each 10 μg of the FGF1, CPPF1, CPPF2, CPPF3, and CPPF4 was diluted with the 0.5 ml of saline containing 5% mouse serum and was administered to the abdominal cavity of the mouse. After the elapse of three and half days from the irradiation, a BrdU labeling solution was injected intraperitoneally and BrdU was incorporated into the dividing cells. After the elapse of two hours, the mice were euthanized, and the jejuna were sampled. After fixing the jejuna with 10% formalin, the paraffin-embedded sections were made. The immunohistostaining was performed on this section with an anti-BrdU antibody, and then this section was stained with hematoxylin.

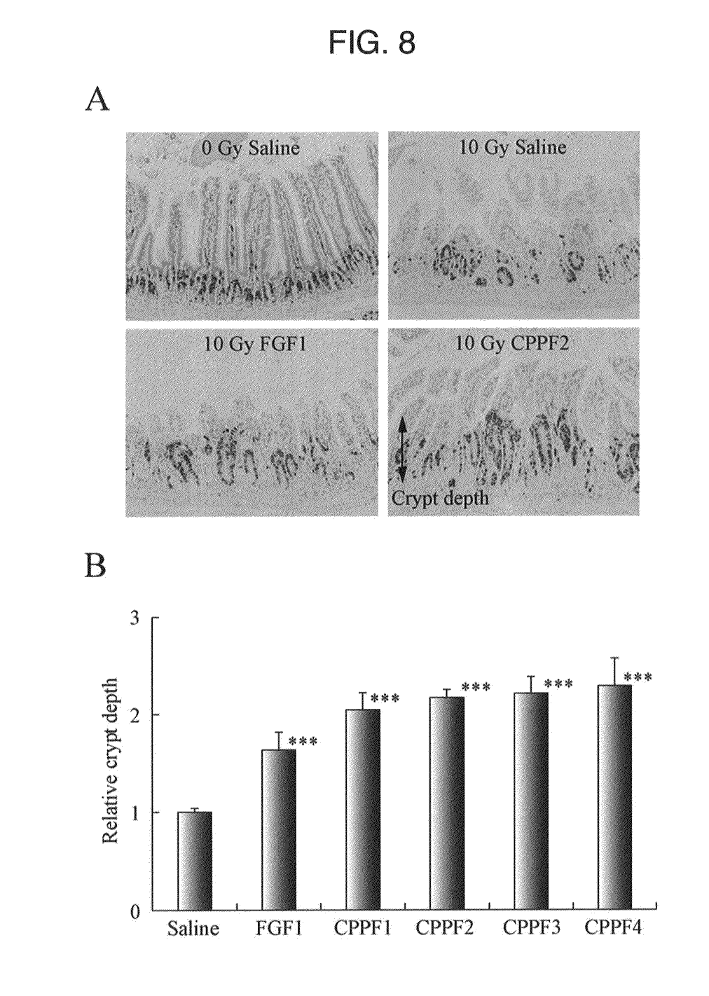

FIG. 8A includes photomicrographies of small intestinal epitheliums on which the immunohistostaining was performed to show crypts with the cells labeled with BrdU and bound by anti-BrdU antibody in each group. Using the microscope, three tissue images were acquired from each group. In each image, the length of ten crypts was measured to obtain the average value of the length in each group. Based on this average value, the relative value was calculated compared with that of the control group administrating the saline. FIG. 8B shows an average relative value+/− standard deviation (S.D.) of the crypt length in each group. In the drawing, *** indicates the experimental groups meeting P<0.001 by the multiple test on the control group.

In the FGF1 administration group, the crypt in the jejunum was significantly longer than in the control group at 3.5 days after total body irradiation of gamma rays at 10 Gy. Meanwhile, in the groups administrating each CPP-FGF1 chimeric protein (CPPF1, CPPF2, CPPF3, and CPPF4), the crypt in the jejunum was not only longer by twice or more than in the control group but also significantly longer (P<0.01 to 0.001) than in the FGF1 administration group.

This result also has proved that CPP-C-fused FGF is more effective at promoting recovery from radiation-induced intestinal damage than FGF1.

### Evaluation for Protective Effect on Stem Cell

This test evaluated the radioprotective effect of CPP-FGF chimeric protein on the stem cells present in the hair follicles against radiation.

Backs of male BALB/c mice of 51 to 53 days old after birth were depilated. Thus, the hair follicles in the resting stage were induced to the growth phase. At the elapse of five days after the depilation, in the control group, 0.5 ml of saline containing 5% mouse serum was administered to the abdominal cavity of the mouse. In the experimental groups, each 100 μg of FGF1, CPPF1, CPPF2, CPPF3, and CPPF4 was diluted in 0.5 ml of saline containing 5% mouse serum and was administered to the abdominal cavity of the mouse. After the elapse of 24 hours, the gamma rays at 12 Gy were irradiated at the dose rate of 0.5 Gy/min on the whole body. After the elapse of 24 hours from the irradiation, the mice were euthanized and then the skins were sampled. Afterwards, the skins were fixed with 10% formalin, and the paraffin-embedded sections were made. Then, the immunohistochemical staining was performed on the paraffin-embedded section with an antibody against Keratin 15, which is a marker for hair follicle stem cells.

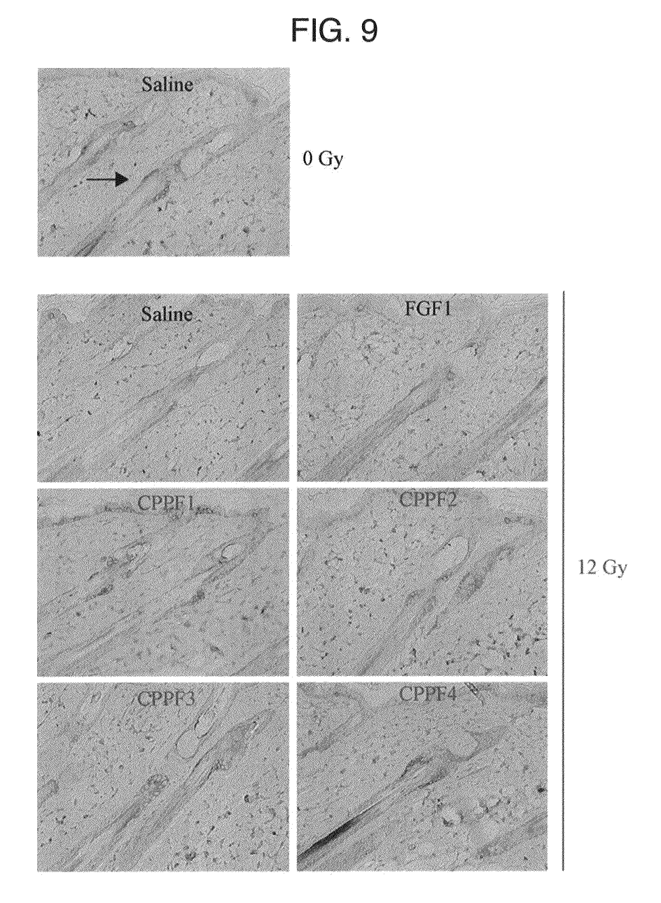

FIG. 9 shows photomicrographies of hair follicle bulge regions stained immunohistochemically in a non-irradiated group, a control group of saline containing 5% mouse serum, and each FGF administration group. The arrow indicates a Keratin 15 positive hair follicle stem cell.

In the control group administering the saline containing 5% mouse serum and irradiated with gamma rays at 12 Gy on the whole body, the Keratin 15 positive hair follicle stem cells in the hair follicle bulge region were reduced compared with the non-irradiated group. In the FGF1 administration group as well, the hair follicle stem cells were reduced by the irradiation. Meanwhile, in the groups administrating the CPP-FGF1 chimeric proteins (CPPF1, CPPF2, CPPF3, and CPPF4), the number of hair follicle stem cells in the bulge region was significantly large compared with not only the control group but also the FGF1 administration group, reaching the number of hair follicle stem cells to a level of equal to or more than the non-irradiation control group. This result has proved that CPP-FGF1 chimeric protein was more effective to protect and maintain the hair follicle stem cells against radiation than FGF1.

### Evaluation for Cellular Internalization Ability 2

This test evaluated the cellular internalization ability of the CPP-FGF1 chimeric protein into cancer cells.

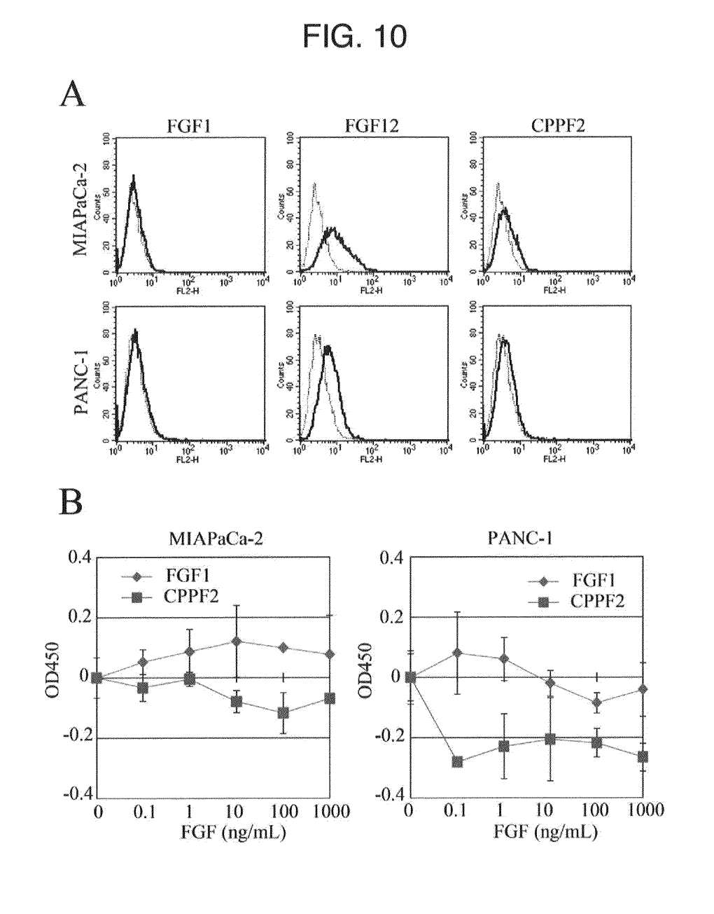

Using human pancreatic cancer cell lines MIAPaCa-2 and PANC-1, the cellular internalization ability of the FGF1, FGF12, and CPPF2 into both cells was measured by the procedure similar to the procedure described in “Evaluation for Cellular Internalization Ability 1.” As illustrated in FIG. 10A, the FGF1 was not able to be translocated into both cells, and the FGF12 also exhibited little cellular internalization. Meanwhile, the CPP-C-fused FGF was able to slightly be translocated into the cells.

### Evaluation for Effect on Inhibiting Proliferation of Cancer Cells 1

This test evaluated the effect of CPP-FGF1 chimeric protein to inhibit the proliferation of the cancer cells utilizing a decomposition by WST-1 cells. The WST-1, which is stable tetrazolium salt, is decomposed into soluble formazans on a surface of a cell having metabolism activity. Accordingly, the WST-1 directly correlates to the number of cells having the metabolism activity during culture. Therefore, the amount of formazan before and after administrating each FGF was measured at an absorbance of 450 nm to evaluate the effect to inhibit the tumor cell proliferation.

In a 96-well plate, 1×10⁴ cells of the human pancreatic cancer cell lines MIAPaCa-2 and PANC-1 were each plated respectively, and were cultured in DMEM culture medium containing 10% FCS for six hours. Afterwards, at a concentration of 5 μg/ml, the heparin was added into the culture medium. In the control group, the FGF was not added while in the experimental groups, the FGF1 and the CPPF2 were further added to the culture medium at the concentration of 0.1 to 1000 ng/mL for each to design the culture medium to be 0.1 mL. The tests were conducted in triplicate for each group. The plate was put into an incubator under an atmosphere at 37° C. and 5% CO₂ and then was cultured for 18 hours. Then, 10 uL of WST-1 reagent (manufactured by Roche Applied Science) was added into the culture medium and was additionally cultured for 4 hours. Afterwards, the absorbance of OD450 was measured to evaluate the tumor cell proliferation.

FIG. 10B illustrates graphs showing the relationship between concentrations of the FGF1 and the CPPF2 and the amount of formazan that increases in association with the cell proliferation. The vertical axis indicates the difference in absorbance from the OD450 value of the control. Accordingly, a higher value means high level of the cell proliferation with respect to the control group.

Adding the FGF1 to the MIAPaCa-2 cells increased the absorbance and increased the tumor cells compared with the control, whereas adding the CPPF2 inhibited the cell proliferation at the concentration of 10 ng/mL or more compared with the control. Meanwhile, although 0.1 to 1 ng/mL FGF1 increased the PANC-1 cells more than the control, the addition of even FGF1 at 100 ng/mL or more inhibited the cell proliferation more than the control. The CPPF2 markedly inhibited the cells at 0.1 ng/mL more than the control, and inhibited the cell proliferation up to 1000 ng/mL. This result has proved that the CPP-FGF1 chimeric protein can inhibit the proliferation of the pancreatic cancer cells.

### Evaluation for Effect on Inhibiting Proliferation of Cancer Cells 1

This test evaluated the effect of CPP-FGF1 chimeric protein to inhibit the proliferation of the cancer cells by colony-forming assay.

In each 6-cm dish, 100 cells of the human pancreatic cancer cell line PANC-1 cells were plated. A DMEM culture medium containing 10% FCS and 5 μg/ml of heparin was added to the dish. The FGF was not added in the control group, while each FGF was further added so as to be 100 ng/ml in the experimental groups. Then, the culture medium of each group was cultured for 13 days. Afterwards, they were fixed with 1% methylene blue/30% methanol and stained, and the number of colonies consisting of 50 cells or more stained on the dish in each group was calculated to evaluate the proliferation ability of the cancer cells.

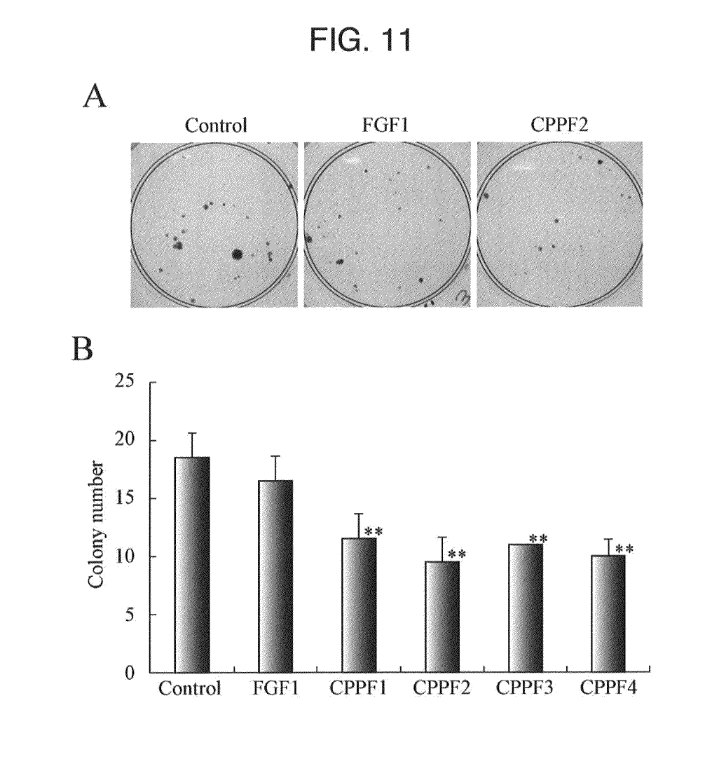

The tests were conducted in duplicate for each group. The average number of colonies on the two dishes in each group was obtained. FIG. 11A includes photographs showing culture plates after being stained in a control group and groups with addition of each FGF. FIG. 11B shows an average value+/− standard deviation (S.D.) of the number of colonies of each group. In the drawing, ** indicates the experimental groups meeting P<0.01 by the multiple test on the control group.

In control group without addition of the FGF, the number of colonies (the average value) reached to 18.5. In the group with addition of FGF1, the number of colonies (the average value) did not significantly reduce compared with the control group. Meanwhile, in the groups with addition of the CPP-FGF1 chimeric proteins (CPPF1, CPPF2, CPPF3, and CPPF4), the number of colonies significantly reduced to 11.5, 9.5, 11, and 10, respectively compared with the control group. A colony reduction ratio to the control group was 37.8% in the CPPF1 administration group, 48.6% in the CPPF2 administration group, 40.5% in the CPPF3 administration group, and 45.9% in the CPPF4 administration group. Thus, in all the groups with addition of the CPP-FGF1 chimeric proteins, the number of colonies reduced to about 40%. Compared with the group with addition of FGF1, in the group with addition of the CPP-FGF1 chimeric protein, the number of colonies significantly reduced (P<0.05 to 0.01). This result has proved that the CPP-FGF1 chimeric protein can significantly inhibit the proliferation of cancers compared with FGF1.

### Evaluation for Effect on Inhibiting Proliferation of Cancer Cells 2

This test evaluated the effect of CPP-FGF1 chimeric protein to inhibit mass formation of the cancer cells using a mouse transplant model.

Experiments using mice were conducted based on the preliminary approved Animal Protocol considering the animal ethics. 1×10⁶ cells of human pancreatic cancer cell lines MIAPaCa-2 were suspended into 10 μl of phosphate buffered saline (PBS) and were injected subcutaneously into the right thigh of a male SCID mouse of seven-week old after birth. In the control group, the mouse was given intraperitoneally 0.5 ml of saline containing 5% mouse serum to the mouse in total of six times, after the elapse of one hour, after the elapse of 24 hours, after the elapse of 48 hours, after the elapse of seven days, after the elapse of 14 days, and after the elapse of 21 days. In the experimental group, 10 μg of CPP-FGF1 chimeric protein (CPPF2) diluted in 0.5-ml of saline containing 5% mouse serum was given. After subcutaneously injecting the cancer cell lines, the size of the subcutaneous mass was measured by digital calipers over time to calculate the average volume in five mice per group.

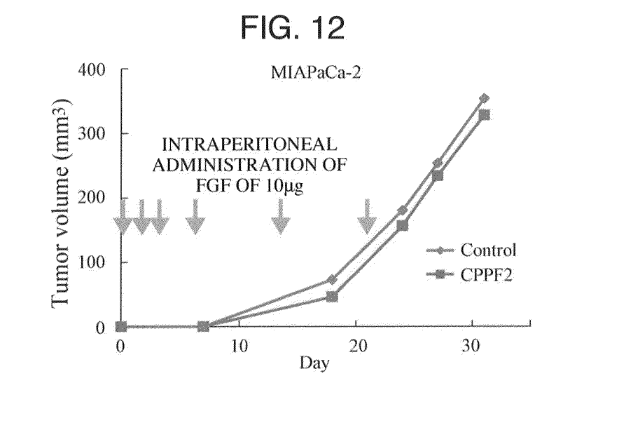

FIG. 12 shows volume changes in a subcutaneous tumor at a right thigh over time in the control group and the experimental group. In the drawing, the arrows indicate the timings of intraperitoneal administrations. In the experimental group administering the CPPF2, the average volume of tumors was always smaller than that in the control group from the 18th day until the 31th day. This result has showed that the CPP-FGF1 chimeric protein has an inhibitory effect on the formation of masses of cancer cells.

### Evaluation of Inhibitory Effect on Metastasis of Cancer Cells

This test evaluated the effect of CPP-FGF1 chimeric protein to inhibit the metastasis of cancer cells using an invasion assay. The cancer cells have the following properties that they secrete protease, break basement membranes, and migrate, when they metastasize. The inhibitory effect of CPP-FGF1 chimeric protein on invasion of cancer cells was evaluated by invasion assay utilizing these properties of cancer cells.

On a filter of a Boyden Chamber for the 24-well plate, 20 μL, (66 μg) of matrigel was coated and gelatinized. Afterwards, to the lower well, 650 μL of DMEM culture medium containing 10% FCS was added. Meanwhile, 1.5×10⁵ cells of the MIAPaCa-2 cells or the PANC-1 cells were suspended into 100 μL of DMEM culture medium containing 0.35% BSA. Then, this suspended liquid was added to the upper well.

Next, 5 μg/ml heparin was added to the culture medium in the lower well and the upper well. To the well of the control group, the FGF was not added. To the wells of the experimental groups, FGF1 and the CPPF2 were each added additionally so as to be 100 ng/mL. The plate was put into an incubator under the atmosphere at 37° C. and 5% CO2 and was cultured for 24 hours. Thus, the invasion of the cancer cells to the gels was induced. The invaded cells were fixed and stained by the Diff-Quick (manufactured by Sysmex Corporation) by the filter of the chamber. The number of stained cells was calculated and regarded as the number of invaded cells. The tests were conducted quadruply for each group and the average value was obtained. A proportion of the average number of invaded cells in each group to the number of cells suspended into the culture medium was obtained and regarded as an invasion cell proportion.

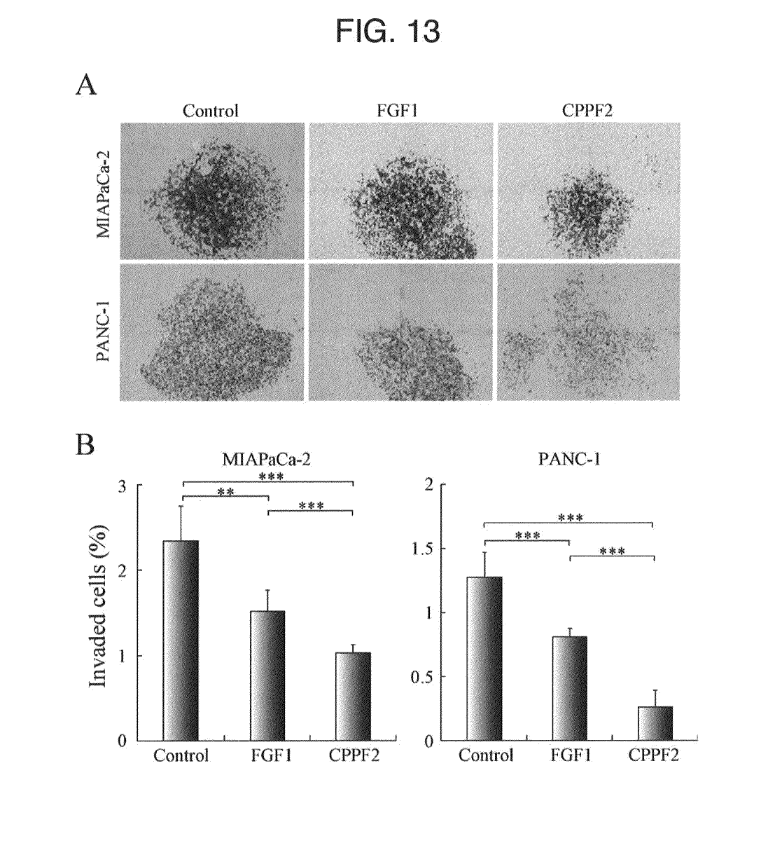

FIG. 13A includes photomicrographies of filters when fixing and staining the cells invaded to the gels by an invasion assay by the Diff-Quick. FIG. 13B shows an average value+/− standard deviation (S.D.) of the invasion cell proportion of each group. In the drawing, ** indicates the experimental group where P<0.01 was met by the multiple test on the control group. *** indicates the experimental groups where P<0.001 was met.

In the control group, the MIAPaCa-2 cells invaded by 2.34%, in the FGF1 administration group, the MIAPaCa-2 cells invaded by 1.52%, and in the CPPF2 administration group, the MIAPaCa-2 cells invaded by 1.03%. In the control group, the PANC-1 cells exhibited the invasion rate of 1.27%, in the FGF1 administration group, the PANC-1 cells exhibited the invasion rate of 0.81%, and in the CPPF2 administration group, the PANC-1 cells exhibited the invasion rate of 0.26%. Seeing this from a perspective of a reduction ratio of invasion with respect to the control group, the FGF1 inhibited the cancer cell invasion to 35% by the MIAPaCa cells and to 36% by the PANG 1. Meanwhile, the CPP-C-fused FGF (CPPF2) further powerfully inhibited the cancer cell invasion, 56% by the MIAPaCa cells and 80% by the PANC-1. This has proved that the CPP-FGF1 chimeric protein inhibited invasive capacity of the cancer cells compared with the FGF1, further inhibiting the metastasis of the cancer.

1. A chimeric protein comprising:
a fibroblast growth factor 1 (hereinafter referred to as an FGF1) or a fibroblast growth factor 2 (hereinafter referred to as an FGF2); and
a cell-membrane peptide (hereinafter referred to as a CPP-C) including an amino acid sequence in a cell-membrane domain present in any of C-terminal regions of fibroblast growth factors 11 to 14 (hereinafter respectively referred to as an FGF11, an FGF12, an FGF13, and an FGF14), or an amino acid sequence having a sequence identity of 60% or more to the amino acid sequence in the cell-membrane domain, the amino acid sequence having a sequence pattern common to a hydrophilic amino acid or a neutral amino acid and a hydrophobic amino acid;
a DNA molecule comprising a DNA sequence coding the FGF1 or the FGF2 and a DNA sequence coding the CPP-C; or
a vector comprising a DNA sequence coding the FGF1 or the FGF2 and a DNA sequence coding the CPP-C.

2. The chimeric protein, the DNA molecule, or the vector according to claim 1 where the FGF1 includes any of following amino acid sequences and an FGF1 activity is maintained:
1) an amino acid sequence is represented by any of SEQ ID NOs. 1 to 5,
2) an amino acid sequence has a sequence identity of 80% or more to an amino acid sequence represented by SEQ ID NO. 1, the amino acid sequence where the amino acids 22 to 28 and 133 in the amino acid sequence are maintained,
3) an amino acid sequence has a sequence identity of 80% or more to an amino acid sequence represented by SEQ ID NO. 2, the amino acid sequence where the amino acids 22 to 28 and 133 in the amino acid sequence are maintained,
4) an amino acid sequence has a sequence identity of 80% or more to an amino acid sequence represented by SEQ ID NO. 3, the amino acid sequence where the amino acids 22 to 28 and 133 in the amino acid sequence are maintained,
5) an amino acid sequence has a sequence identity of 80% or more to an amino acid sequence represented by SEQ ID NO. 4, the amino acid sequence where the amino acids 22 to 28 and 133 in the amino acid sequence are maintained, and
6) an amino acid sequence has a sequence identity of 80% or more to an amino acid sequence represented by SEQ ID NO. 5, the amino acid sequence where the amino acids 22 to 28 and 133 in the amino acid sequence are maintained.

3. The chimeric protein, the DNA molecule, or the vector according to claim 1, wherein
the chimeric protein, the DNA molecule, or the vector has a sequence identity of 90% or more to amino acid sequences 1 to 150 represented by any of SEQ ID NOs. 1 to 5.

4. The chimeric protein, the DNA molecule, or the vector according to claim 1 where the FGF2 includes any of following amino acid sequences and an FGF2 activity is maintained:
1) an amino acid sequence is represented by any of SEQ ID NOs. 6 to 10,
2) an amino acid sequence includes an amino acid sequence where some amino acids in an amino acid sequence represented by SEQ ID NO. 6 are substituted or deleted, or another amino acid is added to the amino acid sequence, the amino acid sequence having a sequence identity of 90% or more to the amino acids 134 to 288 in the amino acid sequence,
3) an amino acid sequence includes an amino acid sequence where some amino acids in an amino acid sequence represented by SEQ ID NO. 7 are substituted or deleted, or another amino acid is added to the amino acid sequence, the amino acid sequence having a sequence identity of 90% or more to the amino acid sequence,
4) an amino acid sequence includes an amino acid sequence where some amino acids in an amino acid sequence represented by SEQ ID NO. 8 are substituted or deleted, or another amino acid is added to the amino acid sequence, the amino acid sequence having a sequence identity of 90% or more to the amino acid sequence,
5) an amino acid sequence includes an amino acid sequence where some amino acids in an amino acid sequence represented by SEQ ID NO. 9 are substituted or deleted, or another amino acid is added to the amino acid sequence, the amino acid sequence having a sequence identity of 90% or more to the amino acid sequence, and
6) an amino acid sequence includes an amino acid sequence where some amino acids in an amino acid sequence represented by SEQ ID NO. 10 are substituted or deleted, or another amino acid is added to the amino acid sequence, the amino acid sequence having a sequence identity of 90% or more to the amino acid sequence.

5. The chimeric protein, the DNA molecule, or the vector according to claim 1, wherein
the CPP-C includes:
1) an amino acid sequence represented by any of SEQ ID NOs. 11 to 29; or
2) an amino acid sequence where some amino acids of the amino acid sequence are substituted, the amino acid sequence having a sequence identity of at least 60% to the amino acid sequence, the amino acid sequence where sequence patterns of hydrophilic amino acids or neutral amino acids and hydrophobic amino acids are common.

6. The chimeric protein, the DNA molecule, or the vector according to claim 1, wherein
the CPP-C includes:
1) an amino acid sequence represented by any of SEQ ID NOs. 11 to 14; or
2) an amino acid sequence where some amino acids of the amino acid sequence are substituted, the amino acid sequence having a sequence identity of at least 60% to the amino acid sequence, the amino acid sequence where sequence patterns of hydrophilic amino acids or neutral amino acids and hydrophobic amino acids are common.

7. The chimeric protein, the DNA molecule, or the vector according to claim 5, wherein
the CPP-C includes an amino acid sequence formed of following amino acids:
1st: proline or leucine
2nd: isoleucine or leucine
3rd: glutamic acid or lysine
4th: valine
5th: cysteine or alanine
6th: methionine or valine
7th: tyrosine
8th: arginine, lysine, or glutamine
9th: glutamic acid.
10th: proline.

8. The chimeric protein, the DNA molecule, or the vector according to claim 1, wherein
the CPP-C is consisted of amino acids of 40 or less.

9. The chimeric protein, the DNA molecule, or the vector according to claim 8, wherein
the CPP-C is consisted of consecutive amino acids of 25 or less derived from any of FGF11, FGF12, FGF13, and FGF14.

10. The medicinal composition according to claim 1, wherein
the CPP-C is bound to or inserted into a C-terminal region of the FGF1 directly or via a joining segment.

11. The chimeric protein, the DNA molecule, or the vector according to claim 1, wherein
the chimeric protein includes an amino acid sequence having a sequence identity of at least 90% to an amino acid sequence represented by any of SEQ ID NOs. 30 to 33.

12. The chimeric protein, the DNA molecule, or the vector according to claim 11, wherein
the chimeric protein maintains the amino acids 22 to 28 and 133 in an amino acid sequence represented by any of SEQ ID NOs. 30 to 33.

13. A composition for medicine or for cell culture, wherein
the composition contains the chimeric protein, the DNA molecule, or the vector according to claim 1.

14. The medicinal composition according to claim 13, wherein
the medicinal composition contains a pharmaceutically acceptable carrier.

15. The chimeric protein, the DNA molecule, the vector, or the composition according to claim 1, wherein
the chimeric protein, the DNA molecule, the vector, or the composition is used to:
maintain or grow a cell,
protect a stem cell,
inhibit an apoptosis of a cell,
promote a migration of a cell,
inhibit a proliferation or a metastasis of a tumor cell, or
recover a function of an ischemic tissue.

16. The chimeric protein, the DNA molecule, the vector, or the composition according to claim 1, wherein
the chimeric protein, the DNA molecule, the vector, or the composition is used to:
promote a healing of a wound,
protect a stem cell against a treatment causing an extinction of a stem cell, such as an exposure to radiation or a chemotherapy,
prevent or treat a disorder of a tissue by radiation,
prevent or treat an ischemic disease, or
treat a malignant tumor.

17. The chimeric protein, the DNA molecule, the vector, or the composition according to claim 1, wherein
the chimeric protein, the DNA molecule, the vector, or the composition is used to:
prevent or treat a damage of an intestinal tract by radiation,
prevent or treat a damage of a hair follicle by radiation or a chemotherapy,
prevent or treat a limb ischemia,
prevent or treat an ischemic coronary artery disease,
prevent or treat a diabetic skin ulcer or a diabetic gangrene,
treat a tympanic membrane perforation, or
inhibit a proliferation or a metastasis of a malignant tumor.

18. A method for preventing or treating a disease or a symptom caused by a physiological phenomenon involving an FGF1 or an FGF2, comprising
a step of administrating the chimeric protein, the DNA molecule, the vector, or the composition according to claim 1 of a therapeutically effective amount to a target requiring the chimeric protein, the DNA molecule, the vector, or the composition.

19. The method according to claim 18, wherein
the method is to:
maintain or grow a cell,
protect a stem cell,
inhibit an apoptosis of a cell,
promote a migration of a cell,
inhibit a proliferation or a metastasis of a tumor cell, or
recover a function of an ischemic tissue.

20. The method according to claim 18, wherein
the method is to:
promote a healing of a wound,
protect a stem cell against a treatment causing an extinction of a stem cell, such as an exposure to radiation or a chemotherapy,
prevent or treat a damage of a tissue by radiation,
prevent or treat an ischemic disease, or
treat a malignant tumor.

21. The method according to claim 18, wherein
the method is to:
prevent or treat a damage of an intestinal tract by radiation,
prevent or treat a disorder of a hair follicle by radiation or a chemotherapy,
prevent or treat a limb ischemia,
prevent or treat an ischemic coronary artery disease,
prevent or treat a diabetic skin ulcer or a diabetic gangrene,
treat a tympanic membrane perforation, or
inhibit a proliferation or a metastasis of a malignant tumor.

22. A use of the chimeric protein, the DNA molecule, the vector, or the composition according to claim 1 to prepare a medicine to:
maintain or grow a cell,
protect a stem cell,
inhibit an apoptosis of a cell,
promote a migration of a cell,
inhibit a proliferation or a metastasis of a tumor cell, or
recover a function of an ischemic tissue.

23. A use of the chimeric protein, the DNA molecule, the vector, or the composition according to claim 1 to prepare a medicine to:
promote a healing of a wound,
protect a stem cell against a treatment causing an extinction of a stem cell, such as an exposure to radiation or a chemotherapy,
prevent or treat a damage of a tissue by radiation,
prevent or treat an ischemic disease, or
treat a malignant tumor.

24. A use of the chimeric protein, the DNA molecule, the vector, or the composition according to claim 1 to prepare a medicine to:
prevent or treat a damage of an intestinal tract by radiation,
prevent or treat a damage of a hair follicle by radiation or a chemotherapy,
prevent or treat a limb ischemia,
prevent or treat an ischemic coronary artery disease,
prevent or treat a diabetic skin ulcer or a diabetic gangrene,
treat a tympanic membrane perforation, or
inhibit a proliferation or a metastasis of a malignant tumor.


SEQUENCE LISTING (34 sequences)

SEQ ID NO: 1 - protein, 155 aa, Homo sapiens
  misc_feature: hFGF1
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 2 - protein, 155 aa, mouse
  misc_feature: mFGF1
MAEGEITTFAALTERFNLPLGNYKKPKLLYCSNGGHFLRILPDGTVDGTRDRSDQHIQLQLSAESAGEVYIKGTETGQYLAMDTEGLLYGSQTPNEECLFLERLEENHYNTYTSKKHAEKNWFVGLKKNGSCKRGPRTHYGQKAILFLPLPVSSD

SEQ ID NO: 3 - protein, 155 aa, rat; identical to SEQ ID NO: 2
  misc_feature: rFGF1

SEQ ID NO: 4 - protein, 155 aa, Bovine
  misc_feature: bFGF1
MAEGETTTFTALTEKFNLPLGNYKKPKLLYCSNGGYFLRILPDGTVDGTKDRSDQHIQLQLCAESIGEVYIKSTETGQFLAMDTDGLLYGSQTPNEECLFLERLEENHYNTYISKKHAEKHWFVGLKKNGRSKLGPRTHFGQKAILFLPLPVSSD

SEQ ID NO: 5 - protein, 155 aa, Equine
  misc_feature: eFGF1
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYTSKKHAEKNWFVGLKKNGSCKRGPRTHYGQKAILFLPLPVSSD

SEQ ID NO: 6 - protein, 288 aa, Homo sapiens
  misc_feature: hFGF2
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 7 - protein, 154 aa, mouse
  misc_feature: mFGF2
MAASGITSLPALPEDGGAAFPPGHFKDPKRLYCKNGGFFLRIHPDGRVDGVREKSDPHVKLQLQAEERGVVSIKGVCANRYLAMKEDGRLLASKCVTEECFFFERLESNNYNTYRSRKYSSWYVALKRTGQYKLGSKTGPGQKAILFLPMSAKS

SEQ ID NO: 8 - protein, 154 aa, rat
  misc_feature: rFGF2
MAAGSITSLPALPEDGGGAFPPGHFKDPKRLYCKNGGFFLRIHPDGRVDGVREKSDPHVKLQLQAEERGVVSIKGVCANRYLAMKEDGRLLASKCVTEECFFFERLESNNYNTYRSRKYSSWYVALKRTGQYKLGSKTGPGQKAILFLPMSAKS

SEQ ID NO: 9 - protein, 155 aa, Bovine
  misc_feature: bFGF2
MAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYSSWYVALKRTGQYKLGPKTGPGQKAILFLPMSAKS

SEQ ID NO: 10 - protein, 155 aa, equine; identical to SEQ ID NO: 9
  misc_feature: eFGF2

SEQ ID NO: 11 - protein, 10 aa, Homo sapiens
  misc_feature: hFGF11CPP-C
LLEVAMYQEP

SEQ ID NO: 12 - protein, 10 aa, Homo sapiens
  misc_feature: hFGF12CPP-C
PIEVCMYREP

SEQ ID NO: 13 - protein, 10 aa, Homo sapiens
  misc_feature: hFGF13CPP-C
PLKVAMYKEP

SEQ ID NO: 14 - protein, 10 aa, Homo sapiens
  misc_feature: hFGF14CPP-C
PLEVAMYREP

SEQ ID NO: 15 - protein, 10 aa, Mouse
  misc_feature: mFGF11CPP-C
LLEVAMYREP

SEQ ID NO: 16 - protein, 10 aa, Mouse
  misc_feature: mFGF12CPP-C
PIEVCMYREP

SEQ ID NO: 17 - protein, 10 aa, Mouse
  misc_feature: mFGF13CPP-C
PLKVAMYKEP

SEQ ID NO: 18 - protein, 10 aa, Mouse
  misc_feature: mFGF14CPP-C
PLEVAMYREP

SEQ ID NO: 19 - protein, 10 aa, Rat
  misc_feature: rFGF11CPP-C
LLEVAVYREP

SEQ ID NO: 20 - protein, 10 aa, Rat
  misc_feature: rFGF12CPP-C
PIEVCMYREP

SEQ ID NO: 21 - protein, 10 aa, Rat
  misc_feature: rFGF13CPP-C
PLKVAMYKEP

SEQ ID NO: 22 - protein, 10 aa, Rat
  misc_feature: rFGF14CPP-C
PLEVAMYREP

SEQ ID NO: 23 - protein, 10 aa, Bovine
  misc_feature: bFGF11CPP-C
PIEVCMYREP

SEQ ID NO: 24 - protein, 10 aa, Bovine
  misc_feature: bFGF12CPP-C
PIEVCMYREP

SEQ ID NO: 25 - protein, 10 aa, Bovine
  misc_feature: bFGF13CPP-C
PLKVAMYKEP

SEQ ID NO: 26 - protein, 10 aa, Bovine
  misc_feature: bFGF14CPP-C
PLEVAMYREP

SEQ ID NO: 27 - protein, 10 aa, Equine
  misc_feature: eFGF11CPP-C
PIEVCMYREP

SEQ ID NO: 28 - protein, 10 aa, Equine
  misc_feature: eFGF13CPP-C
PLKVAMYKEP

SEQ ID NO: 29 - protein, 10 aa, Equine
  misc_feature: eFGF14CPP-C
PLEVAMYREP

SEQ ID NO: 30 - protein, 169 aa, Artificial Sequence
  CPPF1
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLEFLLEVAMYQEPVDPVSSD

SEQ ID NO: 31 - protein, 169 aa, Artificial Sequence
  CPPF2
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLEFPIEVCMYREPVDPVSSD

SEQ ID NO: 32 - protein, 169 aa, Artificial Sequence
  CPPF3
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLEFPLKVAMYKEPVDPVSSD

SEQ ID NO: 33 - protein, 169 aa, Artificial Sequence
  CPPF4
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLEFPLEVAMYREPVDPVSSD

SEQ ID NO: 34 - protein, 181 aa, Homo sapiens
  misc_feature: hFGF12B
MESKEPQLKGIVTRLFSQQGYFLQMHPDGTIDGTKDENSDYTLFNLIPVGLRVVAIQGVKASLYVAMNGEGYLYSSDVFTPECKFKESVFENYYVIYSSTLYRQQESGRAWFLGLNKEGQIMKGNRVKKTKPSSHFVPKPIEVCMYREPSLHEIGEKQGRSRKSSGTPTMNGGKVVNQDST